SEQUENCE LISTING (15 sequences)

SEQ ID NO: 1 - DNA, 21 bp, Artificial Sequence
  Artificially synthesized oligonucleotide primer
agctaaccacaccttataccg

SEQ ID NO: 2 - DNA, 22 bp, Artificial Sequence
  Artificially synthesized oligonucleotide primer
ggtgaaggtcggagtcaacgga

SEQ ID NO: 3 - DNA, 22 bp, Artificial Sequence
  Artificially synthesized oligonucleotide primer
gagggatctcgctcctggaaga

SEQ ID NO: 4 - DNA, 20 bp, Artificial Sequence
  Artificially synthesized oligonucleotide primer
gcccatcctctgtgactcat

SEQ ID NO: 5 - DNA, 20 bp, Artificial Sequence
  Artificially synthesized oligonucleotide primer
aggccacaggtattttgtcg

SEQ ID NO: 6 - DNA, 20 bp, Artificial Sequence
  Artificially synthesized oligonucleotide primer
aagtttgccttgaccctgaa

SEQ ID NO: 7 - DNA, 20 bp, Artificial Sequence
  Artificially synthesized oligonucleotide primer
aggcacatcaggtacgatcc

SEQ ID NO: 8 - DNA, 20 bp, Artificial Sequence
  Artificially synthesized oligonucleotide primer
tccagaaggccctcagacta

SEQ ID NO: 9 - DNA, 20 bp, Artificial Sequence
  Artificially synthesized oligonucleotide primer
acacccaccagcatcttctc

SEQ ID NO: 10 - DNA, 24 bp, Artificial Sequence
  Artificially synthesized oligonucleotide primer
gaagttcaacatcaagagcagtag

SEQ ID NO: 11 - DNA, 20 bp, Artificial Sequence
  Artificially synthesized oligonucleotide primer
agggagaagtaggaatgggg

SEQ ID NO: 12 - DNA, 21 bp, Artificial Sequence
  Artificially synthesized oligonucleotide primer
atgagcacagaaagcatgatc

SEQ ID NO: 13 - DNA, 21 bp, Artificial Sequence
  Artificially synthesized oligonucleotide primer
tacaggcttgtcactcgaatt

SEQ ID NO: 14 - DNA, 5379 bp, Homo sapiens
gcacttccttctcggctagattatctgaaactgttgtcggttcttgagatgatactaccaccgaatgtctgtgtttcattgtctagtccaacctgtattgtggatatctacaacgttccggcaatagttttgcaggtgcatcacatttttgtttttgttttgggaggaaaagggagggcacggcagccaggcttcatattcctacaagtgcatgcttcaagattactgtacttacagtgtttccaacatcttctcataaaaggggaaagcttcatagcctcaaccatgaaggaaaccagtcgcatagggcatggagctggagaactataaacagcccgtggtgctgagagaggacaactgccgaaggcgccggaggatgaagccgcgcagtgctgcggccagcctgtcctccatggagctcatccccatcgagttcgtgctgcccaccagccagcgcaaatgcaagagccccgaaacggcgctgctgcacgtggccggccacggcaacgtggagcagatgaaggcccaggtgtggctgcgagcgctggagaccagcgtggcggcggacttctaccaccggctgggaccgcatcacttcctcctgctctatcagaagaaggggcagtggtacgagatctacgacaagtaccaggtggtgcagactctggactgcctgcgctactggaaggccacgcaccggagcccgggccagatccacctggtgcagcggcacccgccctccgaggagtcccaagccttccagcggcagctcacggcgctgattggctatgacgtcactgacgtcagcaacgtgcacgacgatgagctggagttcacgcgccgtggcttggtgaccccgcgcatggcggaggtggccagccgcgaccccaagctctacgccatgcacccgtgggtgacgtccaagcccctcccggagtacctgtggaagaagattgccaacaactgcatcttcatcgtcattcaccgcagcaccaccagccagaccattaaggtctcacccgacgacacccccggcgccatcctgcagagcttcttcaccaagatggccaagaagaaatctctgatggatattcccgaaagccaaagcgaacaggattttgtgctgcgcgtctgtggccgggatgagtacctggtgggcgaaacgcccatcaaaaacttccagtgggtgaggcactgcctcaagaacggagaagagattcacgtggtactggacacgcctccagacccggccctagacgaggtgaggaaggaagagtggccgctggtggatgactgcacgggagtcaccggctaccatgagcagcttaccatccacggcaaggaccacgagagtgtgttcaccgtgtccctgtgggactgcgaccgcaagttcagggtcaagatcagaggcattgatatccccgtcctgcctcggaacaccgacctcacagtttttgtagaggcaaacatccagcatgggcaacaagtcctttgccaaaggagaaccagccccaaacccttcacagaggaggtgctgtggaatgtgtggcttgagttcagtatcaaaatcaaagacttgcccaaaggggctctactgaacctccagatctactgcggtaaagctccagcactgtccagcaaggcctctgcagagtcccccagttctgagtccaagggcaaagttcagcttctctattatgtgaacctgctgctgatagaccaccgtttcctcctgcgccgtggagaatacgtcctccacatgtggcagatatctgggaagggagaagaccaaggaagcttcaatgctgacaaactcacgtctgcaactaacccagacaaggagaactcaatgtccatctccattcttctggacaattactgccacccgatagccctgcctaagcatcagcccacccctgacccggaaggggaccgggttcgagcagaaatgcccaaccagcttcgcaagcaattggaggcgatcatagccactgatccacttaaccctctcacagcagaggacaaagaattgctctggcattttagatacgaaagccttaagcacccaaaagcatatcctaagctatttagttcagtgaaatggggacagcaagaaattgtggccaaaacataccaattgttggccagaagggaagtctgggatcaaagtgctttggatgttgggttaacaatgcagctcctggactgcaacttctcagatgaaaatgtaagagccattgcagttcagaaactggagagcttggaggacgatgatgttctgcattaccttctacaattggtccaggctgtgaaatttgaaccataccatgatagcgcccttgccagatttctgctgaagcgtggtttaagaaacaaaagaattggtcactttttgttttggttcttgagaagtgagatagcccagtccagacactatcagcagaggttcgctgtgattctggaagcctatctgaggggctgtggcacagccatgctgcacgactttacccaacaagtccaagtaatcgagatgttacaaaaagtcacccttgatattaaatcgctctctgctgaaaagtatgacgtcagttcccaagttatttcacaacttaaacaaaagcttgaaaacctgcagaattctcaactccccgaaagctttagagttccatatgatcctggactgaaagcaggagcgctggcaattgaaaaatgtaaagtaatggcctccaagaaaaaaccactatggcttgagtttaaatgtgccgatcctacagccctatcaaatgaaacaattggaattatctttaaacatggtgatgatctgcgccaagacatgcttattttacagattctacgaatcatggagtctatttgggagactgaatctttggatctatgcctcctgccatatggttgcatttcaactggtgacaaaataggaatgatcgagattgtgaaagacgccacgacaattgccaaaattcagcaaagcacagtgggcaacacgggagcatttaaagatgaagtcctgaatcactggctcaaagaaaaatcccctactgaagaaaagtttcaggcagcagtggagagatttgtttattcctgtgcaggctactgtgtggcaacctttgttcttggaataggcgacagacacaatgacaatattatgatcaccgagacaggaaacctatttcatattgacttcgggcacattcttgggaattacaaaagtttcctgggcattaataaagagagagtgccatttgtgctaacccctgacttcctctttgtgatgggaacttctggaaagaagacaagcccacacttccagaaatttcaggacatctgtgttaaggcttatctagcccttcgtcatcacacaaacctactgatcatcctgttctccatgatgctgatgacaggaatgccccagttaacaagcaaagaagacattgaatatatccgggatgccctcacagtggggaaaaatgaggaggatgctaaaaagtattttcttgatcagatcgaagtttgcagagacaaaggatggactgtgcagtttaattggtttctacatcttgttcttggcatcaaacaaggagagaaacattcagcctaatactttaggctagaatcaaaaacaagttagtgttctatggtttaaattagcatagcaatcatcgaacttggatttcaaatgcaatagacattgtgaaagctggcatttcagaagtatagctcttttcctacctgaactcttccctggagaaaagatgttggcattgctgattgtttggttaagcaatgtccagtgctaggattatttgcaggtttggttttttctcatttgtctgtggcattggagaatattctcggtttaaacagactaatgacttccttattgtccctgatattttgactatcttactattgagtgcttctggaaattctttggaataattgatgacatctattttcatctgggtttagtctcaattttggttatctttgtgttcctcaagctctttaaagaaaaagatgtaatcgttgtaacctttgtctcattccttaaatgatgcttccaaacatctccttagtgtctgcaggtgttagtggtgtgctaaaagcaaggaaagcgagttagtcttttcagtgtcttttgcaattcaattcttttgtcatgtataactgagacacacaaacacagcaggagaaatctaaaccgttgtgccttgaccttcctctgctggtcttgttccagggttatgaatatgaaaaaatagagatgagactttttgtgtcaactctgtccacaagagtgagttatctagtatgattagtatagctttctccagcatggcagcaggaagtaactacagggcctcttttatgcctgacatttcttcccttcctttttccctgcctccctttttcatcaattgcaatgctcccacaactctttacagacttgtgaaatcttcaagaacacctttactctataactcaaaaattagttgaaaaataattacttctcaaggattattagaatcttaggtacttatttgtaaagatgtttagtgacttttttttcaagtatcttattaaaggaggcattctagaaaatatgaattagtttccaaatgccttaattttaaactttggcctgaacagttttttctttttcttaatggaagaagatatttaatatcttaaaaatattccaagttaggaagaacactacttgccttatccatttcccatttaaaggacttttaaactttgacacatccttcagatttcctgaaaataattgaaatatcttactttaaaaatattttcatctctgaaatatctcgttatttattggaggtattgtttaaccttagagagaccattaaattatttataaaatattttgtaattacctgtagctaatacattacatagaaaaaaactatgttaacagtgtctctgtttaagtataatcagatataaatatatacttaattttttaattttaaaaatagatacctgtttgactttgaggtagtccagaccttttctttttttttttttttttaatgtgtgcaaaagcccaaaggttcctaagcctggctgcaaagaagaatcaacagggacactttttaaaaacactcttatcagcctgggcaacacagtgagactccatctcttaaaaaaaaaattagctgggtatagtggtatgtgcctgtagtcccaggtactcaggaggctgaggcaggaggattgcctgagcccaggaggtggaaactgcagagagtcatgatcatgtccttacactccagcctggataacagagcgagaccctgtctcaaaaaaataaaataaaaaataaaaaca

SEQ ID NO: 15 - DNA, 8733 bp, Homo sapiens
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgtaaaaaaaaa

(19) United States
(12) Patent Application Publication
(10) Pub. No.: US 2013/0225629 A1
(43) Pub. Date: Aug. 29, 2013

(54) TARGETING PI3K/MTOR SIGNALLING AND NEUTROPHIL RECRUITMENT FOR TREATMENT OF ENTERITIS

(71) Applicant: THE UNIVERSITY OF NORTH CAROLINA AT CHAPEL HILL, (US)

(72) Inventors: Christian Jobin, Hillsborough, NC (US); Xiaolun Sun, Chapel Hill, NC (US)

(73) Assignee: THE UNIVERSITY OF NORTH CAROLINA AT CHAPEL HILL, Chapel Hill, NC (US)

(21) Appl. No.: 13/776,228

(22) Filed: Feb. 25, 2013

Related U.S. Application Data
(60) Provisional application No. 61/602,933, filed on Feb. 24, 2012.

(51) Int. Cl. A61K 31/436; A61K 31/4178; A61K 31/366

(52) U.S. Cl. CPC A61K 31/436; A61K 31/366; A61K 31/4178
USPC 514/291; 514/453; 514/369; 435/6.13

(57) ABSTRACT

The presently disclosed subject matter generally relates to methods and compositions for treating enteritis. More particularly, the presently disclosed subject matter relates to methods and compositions for modulating a component of a PI3K/mTOR pathway. In some embodiments, the methods and compositions of the presently disclosed subject matter generally relates to the treatment of campylobacteriosis. More particularly, the methods and compositions of the presently disclosed subject matter relate to the treatment of campylobacteriosis by modulating a component of a PI3K/mTOR pathway.

### CROSS REFERENCE TO RELATED APPLICATIONS

This application claims the benefit of U.S. Provisional Patent Application Ser. No. 61/602,933, filed Feb. 24, 2012, the disclosure of which is incorporated herein by reference in its entirety.

### GRANT STATEMENT

This invention was made with government support under grants DK047700, DK073338, AI082319 and P30 DK34987 awarded by NIH. The government has certain rights in the invention.

### TECHNICAL FIELD

The presently disclosed subject matter relates to methods of assessing, modulating, attenuating, reversing and/or treating enteritis, including campylobacteriosis. Also described are agents for modulating, attenuating, reversing and/or treating enteritis, including campylobacteriosis.

### BACKGROUND

Campylobacter jejuni is a gram-negative category B priority food- and water-borne pathogen and the worldwide leading bacterial causative agent of enteritis (Allos, 2001). The Centers for Disease Control and Prevention estimate that 2.4 million subjects are infected with C. jejuni resulting in 124 deaths every year in the United States. Clinical symptoms of C. jejuni infection include abdominal cramps, watery to bloody diarrhea, fever and gastrointestinal inflammation (Blaser et al., 1997). At the cellular level, C. jejuni infected patients display infiltration of immune cells such as neutrophils, crypt abscesses and presence of fecal leukocytes (van Spreeuwel et al., 1985). Although the intestinal disease self-resolves within one week, a small portion of patients (1:1000) develop extra-intestinal sequelae such as Guillain-Barre Syndrome (GBS) and reactive arthritis (Nachamkin, 2002). Interestingly, C. jejuni exposure can also be responsible for initiation and relapse of inflammatory bowel diseases (IBD; Gradel et al., 2009) and post-infectious irritable bowel syndrome (Qin et al., 2010).

Despite the prevalence of C. jejuni induced illness and negative socio-economic impact, little information is available regarding the molecular and cellular events involved in campylobacteriosis. The major contributing factor to the poor understanding of campylobacteriosis is the lack of robust experimental models mimicking the various phases of acute human infection. Although some mammals including monkey (Russell et al., 1989), ferret (Nemelka et al., 2009), and piglet (Law et al., 2009) have provided valuable information regarding cellular events associated with campylobacteriosis, limited reagents and lack of genetic manipulation in these models have constrained the generation of deep mechanistic understanding. Applicants recently established an acute model of campylobacteriosis using germ-free II10^{-/-} mice (Lippert et al., 2009). In this model, C. jejuni induces a rapid (5 days) and robust inflammatory (bloody diarrhea) response to the microorganism. However, the cellular and molecular details responsible for this host response remained undefined.

There remains a need, therefore, for a better understanding of the molecular and cellular events involved in campylobacteriosis, including the cellular and molecular details responsible for the host response. Methods of assessing, modulating, attenuating, reversing and/or treating campylobacteriosis are needed. Agents for modulating, attenuating, reversing and/or treating campylobacteriosis are also needed.

### SUMMARY

The presently disclosed subject matter provides methods of treating enteritis, including campylobacteriosis. Also described are agents to for treating enteritis, including campylobacteriosis.

It is an object of the presently disclosed subject matter to provide a method of treating enteritis in a subject. In some embodiments, a method of treating enteritis in a subject is provided, the method comprising providing a subject suffering from enteritis, and administering to the subject a composition comprising a compound capable of modulating a component of a PI3K pathway, wherein the enteritis is treated. In some embodiments, a causative agent of the enteritis is selected from the group consisting of Campylobacter jejuni, Salmonella typhimurium, Enteropathogenic Escherichia coli and Shigella. In some embodiments, the subject is suffering from campylobacteriosis. In some embodiments, the compound capable of modulating a component of the PI3K pathway comprises an inhibitor of mammalian target of rapamycin (mTOR). In some embodiments, the inhibitor of mTOR is rapamycin, rapamycin derivatives or analogues. In some embodiments, the inhibitor of mTOR is Rapamune, Torisel, Afinitor or Zortress. In some embodiments, the compound capable of modulating a component of the PI3K pathway comprises an inhibitor of PI3K. In some embodiments, the inhibitor of PI3K is wortmannin. In some embodiments, the compound capable of modulating a component of the PI3K pathway comprises an inhibitor of PI3Kγ. In some embodiments, the inhibitor of PI3Kγ is selected from the group consisting of AS252424, thiazolidinones, thiazolidinones, and 2-aminothiazoles. In some embodiments, treating the enteritis comprises reduced intestinal inflammation or increased bacterial clearance. In some embodiments, the subject is a human.

In some embodiments a method of identifying an agent to treat enteritis is provided, the method comprising providing a test sample comprising a polypeptide of a PI3K pathway, administering a test molecule to the test sample, and determining the effect of the test molecule on the activity of the polypeptide of a PI3K pathway. In some embodiments, the polypeptide of the PI3K pathway comprises mTOR complex 1 or mTOR complex 2. In some embodiments, the polypeptide of the PI3K pathway comprises PI3Kγ. In some embodiments, the effect of the test molecule on the activity of the polypeptide of the PI3K pathway is a modulatory effect. In some embodiments, the modulatory effect on the polypeptide of the PI3K pathway is an inhibition of a signaling activity of the PI3K polypeptide.

In some embodiments a therapeutic composition to treat enteritis in a subject is provided, the therapeutic composition comprising a compound capable of modulating a component of a PI3K pathway, and a pharmaceutically acceptable carrier. In some embodiments, the compound capable of modulating a component of the PI3K pathway comprises an inhibitor of mTOR. In some embodiments, the inhibitor of mTOR is rapamycin, rapamycin derivatives or analogues. In some embodiments, the compound capable of modulating a component of the PI3K pathway comprises an inhibitor of PI3K. In some embodiments, the inhibitor of PI3K is wortmannin. In some embodiments, the compound capable of modulating a component of the PI3K pathway comprises an inhibitor of PI3Kγ. In some embodiments, the inhibitor of PI3Kγ is selected from the group consisting of AS252424, thiazolidinones, thiazolidinones, and 2-aminothiazoles.

An object of the presently disclosed subject matter having been stated hereinabove, and which is achieved in whole or in part by the presently disclosed subject matter, other objects will become evident as the description proceeds when taken in connection with the accompanying Examples as best described hereinbelow.

### BRIEF DESCRIPTION OF THE DRAWINGS

FIGS. 1A-1C depict results of studies showing that rapamycin prevents and treats C. jejuni induced colitis in II10^{-/-}; NF-κB^{EGFP} mice. FIG. 1A is a Western blot for total and phosphorylated colonic p70S6. FIG. 1B is a bar graph of histological intestinal damage scores of rapamycin prevention on C. jejuni infection. FIG. 1C is a bar graph of histological intestinal damage score of rapamycin treatment. All graphs depict mean±SE. **P<0.01; ***P<0.001. Results are representative of 4 independent experiments.

FIGS. 2A-2B depict results of studies demonstrating that C. jejuni induced intestinal inflammation is independent of CD4 T cell activation. FIG. 1A is a bar graph showing histological scores of intestinal inflammation. FIG. 1B is a bar graph showing percentage flow cytometry results of CD4+ cell in the spleen and mesenteric lymph nodes (MLN). All graphs depict mean±SE. ***P<0.001. Results are representative of 3 independent experiments.

FIG. 3 depicts results of studies demonstrating that rapamycin treatment reduces C. jejuni induced colonic EGFP expression in II10^{-/-}; NF-κB^{EGFP} mice. FIG. 3 is a Western blot illustrating EGFP levels. Results are representative of 3 independent experiments.

FIGS. 4A and 4B are bar graphs showing that rapamycin attenuates C. jejuni induced expression of proinflammatory mediators. FIG. 4A includes bar graphs of II1β, Cxcl2 and II-17a mRNA accumulation quantified using an ABI 7900HT Fast Real-Time PCR System and specific primers and data were normalized to Gapdh. FIG. 4B includes bar graphs of ELISA results measuring IL-1β and IL-17 secretion in supernatants collected from colonic tissues and mesenteric lymph nodes cultured in RPMI 1640 medium supplemented with 3% FBS and 1% antibiotics for 18 hrs. Data represent means±SE. *P<0.05; **P<0.01. Results are representative of 3 independent experiments.

FIG. 5 depicts the results of experiments that demonstrate that Neutrophils participate in C. jejuni induced colitis. FIG. 5 is a bar graph showing the results of flow cytometry analysis for CD45⁺ and Gr-1⁺ cells in the peripheral blood of infected and rapamycin-treated mice. Data represent means±SE **P<0.01. Results are representative of 3 independent experiments.

FIG. 6 depicts results of studies demonstrating that mTOR signaling promotes C. jejuni invasion of the colon, MLN and spleen. FIG. 6 is a bar graph of showing C. jejuni bacterial count in the stool, colon, MLN and spleen of untreated or rapamycin-treated mice. Data represent means±SE *P<0.05. Results are representative of 3 independent experiments.

FIGS. 7A and 7B depict results of studies demonstrating that rapamycin promotes C. jejuni eradication and LC3II generation in splenocytes. FIG. 7A is a bar graph illustrating the percentage of C. jejuni survival in splenocytes. FIG. 7B is a Western blot of colonic LC3 I/II amd phosphorylated p70S6 in splenocytes.

FIG. 8 depict results of studies demonstrating that rapamycin attenuates C jejuni-induced colitis in conventionally derived II10^{-/-}; NF-κB^{EGFP} mice. FIG. 8 is a bar graph depicting histologic intestinal damage scores of rapamycin prevention on C jejuni infection. The graph depicts mean±SE. *P<0.05. Results are representative of 2 independent experiments.

FIG. 9A-9B illustrate that rapamycin ameliorates S. typhimurium-induced intestinal inflammation in cecum and colon of II10^{-/-}; NF-κB^{EGFP} mice.

FIG. 9A is a bar graph of histologic cecal damage scores of rapamycin-treated, S. typhimurium-infected mice. FIG. 9B is a bar graph of histologic colonic damage scores of rapamycin-treated, S. typhimurium-infected mice. All graphs depict mean±SE. *P<0.05. Results are representative of 2 independent experiments.

FIG. 10 illustrates that C jejuni induces early colitis in II10^{-/-}; NF-κB^{EGFP} mice. FIG. 10 is a bar graph of histologic intestinal damage scores of rapamycin treated, C jejuni-infected mice. Results are representative of 2 independent experiments.

FIGS. 11A and 11B illustrate that C jejuni-induced II-12p40 and TNFα messenger RNA accumulation is not blocked by rapamycin. FIGS. 11A and 11B are bar graphs of II-12p40 (FIG. 13A) and TNFα (FIG. 13B) messenger RNA accumulation quantified using an ABI 7900HT Fast Real-Time PCR System, and specific primers and data were normalized to Gapdh. Data represent means±SE. Results are representative of 3 independent experiments.

FIGS. 12A and 12B illustrate that innate immune cells mediate C. jejuni induced colitis in II10^{-/-} mice. FIG. 12A is a bar graph depicting the quantification of histological intestinal damage scores mediated by C. jejuni infection. FIG. 12B is a series of bar graphs showing the results of quantifying II1β, Cxcl2 and 455 II17a mRNA accumulation using an ABI 7900HT Fast Real-Time PCR System with specific primers and data normalized to Gapdh. Open bars refer to II10^{-/-}, solid bars refer to II10^{-/-}; Rag2^{-/-}. All graphs depict mean±SEM. NS (not significant), P>0.05. Results are representative of 3 independent experiments.

FIGS. 13A-13C illustrate that the PI3K signaling pathway mediates C. jejuni-induced intestinal inflammation in II10^{-/-}; NF-κBEGFP mice. FIG. 13A is a bar graph showing the results of the quantification of intestinal inflammation based on histological scores. FIG. 13B is a Western blot for total and phosphorylated (S473) AKT and EGFP protein levels in pooled colonic lysates of infected mice. FIG. 13C is a series of bar graphs depicting op, Cxcl2 and II17a mRNA accumulation quantified using real time PCR. All graphs depict mean±SEM. *, P<0.05, **, P<0.01, ***, P<0.001. Results are representative of 3 independent experiments.

FIGS. 14A-14D illustrate that pharmacological inhibition of PI3Kγ blocks C. jejuni-induced intestinal inflammation in II10^{-/-}; NF-κBEGFP mice. FIG. 14A is a bar graph depicting quantitative histological scores of intestinal inflammation. FIG. 14B is a Western blot for total and phosphorylated (S473) AKT, phosphorylated p70S6K (T389) and EGFP protein levels in pooled colonic lysates of infected mice. FIG. 14C includes bar graphs depicting the density of Western blot bands was quantified using ImageJ and normalized to control. FIG. 14D is a series of bar graphs showing II1β, Cxcl2 and II17a mRNA accumulation quantified by real time PCR. All graphs depict mean±SEM. *, P<0.05, **, P<0.01, NS, not significant. Results are representative of 3 independent experiments.

FIGS. 15A-15C illustrate that PI3Kγ deficiency attenuates C. jejuni-induced intestinal inflammation. FIG. 15A is a bar graph depicting the quantitative histological score of intestinal inflammation in Wt, II10^{-/-} and Pi3kγ^{-/-} mice, respectively, treated with antibiotic for 7 days and then gavaged with a single dose of C. jejuni (109/mouse). FIG. 15B is a Western blot for phosphorylated AKT (S473), phosphorylated p70S6K (T389) and total AKT protein levels in pooled colonic lysates of infected mice. FIG. 15C is a series of bar graphs depicting II1β, Cxcl2 and II17a mRNA accumulation quantified using real time PCR. Open bars refer to Wt, solid bars refer to PI3Kγ. Data represent means±SEM. *, P<0.05. Scale bar is 200 μm. Results are representative of 2 independent experiments.

FIG. 16 illustrates that PI3Kγ signaling promotes C. jejuni invasion into colon, MLN and spleen. FIG. 16 is a series of bar graphs depicting C. jejuni bacterial count in the colon, MLN and spleen of vehicle- or AS252424-treated mice. Data represent means±SEM. *P<0.05. Results are representative of 3 independent experiments.

FIGS. 17A-17B illustrate that PI3Kγ mediates neutrophil accumulation and crypt abscesses in C. jejuni infected mice. FIG. 17A is a bar graph depicting the number of crypt abscesses in C. jejuni infected mice. FIG. 17B is a bar graph depicting the quantitative measurements of migrated neutrophils. Data represent means±SEM. **, P<0.01. Results are representative of 3 independent experiments.

FIGS. 18A-18B illustrate that neutrophils enhance C. jejuni-induced colitis. FIG. 18A is a bar graph depicting the quantitative histological score of intestinal inflammation. FIG. 18B is a bar graph depicting that the colonic hematoxylin and eosin stained sections were imaged (5 fields/mouse) and neutrophils were identified based on morphological features. Data are presented as average counts/mouse. Data represent means±SEM. *, P<0.05. Results are representative of 3 independent experiments.

FIG. 19 is a schematic illustration of the PI3K/mTOR pathway and the components thereof.

### DETAILED DESCRIPTION

The presently disclosed subject matter is directed in some embodiments to methods of treating enteritis in a subject. Enteritis refers to the inflammation of the intestine, often caused by the ingestion of enteropathic microorganisms. By way of example and not limitation, causative agents of enteritis can include Campylobacter jejuni, Salmonella typhimurium, Enteropathogenic Escherichia coli and Shigella. The presently disclosed subject matter is also directed to methods of identifying agents to treat enteritis. The presently disclosed subject matter is also directed to agents useful in treating enteritis.

The presently disclosed subject matter relates to the discovery that the phosphoinositide 3-kinase (PI3K) pathway, including components of the PI3K pathway, such as for example PI3Kγ, PI3Kp110β, P13 Kp110β, PI3Kp110δ, PI3Kp110α, AKT, S6K1 and mammalian target of rapamycin (mTOR), is involved in a signaling event that regulates a host response to C. jejuni infection. Signaling intermediates in this pathway such as AKT and ribosomal protein S6 kinase beta-1 (S6K1) are also implicated in the regulation of C. jejuni infection (Weichhart and Saemann, 2008). Disclosed herein are findings that demonstrate that PI3K pathway signaling, including for example PI3Kγ and mTOR signaling, regulates C. jejuni induced expression of inflammatory mediators, neutrophil infiltration and bacterial clearance. These molecular and signaling events represent steps in C. jejuni induced intestinal inflammation and define new therapeutic targets.

Phosphatidylinositol 3-kinases (PI3Ks) are a large family of signaling proteins formed by a catalytic subunit and a regulatory subunit. These signaling proteins are grouped into three different classes (I, II and III) and are implicated in the regulation of cell growth, proliferation, differentiation, survival and motility. In addition, various PI3K proteins are implicated in innate and adaptive immunity. PI3Kγ is a class I B PI3K and comprises a catalytic subunit (p110γ) and a regulatory subunit (p101 or p84). PI3Kγ is mainly expressed in immune cells and mediates chemoattractant induced cell migration by controlling actin cytoskeletal rearrangement through G-protein coupled receptors. Interestingly, neutrophils isolated from Pi3kγ^{-/-} mice show impaired migration towards N-formyl-methionyl-leucyl-phenylalanine (fMLP) due to reduced F-actin accumulation at the cell's leading edge (Ferguson et al., 2007). In addition, Pi3kγ^{-/-} mice injected i.p. with Listeria exhibited reduced neutrophil accumulation into the peritonea compared to Wt mice (Sasaki et al., 2000). As stated above, numerous signaling subunits participate in the PI3K pathway and isoforms such as PI3Kp110β, PI3Kp110δ, PI3Kp110α may be implicated in enteritis.

mTOR plays a role in cell growth and proliferation. The mTOR complex is composed of two entities: mTOR complex 1 (mTORC1) and mTOR complex 2 (mTORC2). mTORC1 is a downstream target of PI3K and is sensitive to the pharmacological inhibitor rapamycin, or derivatives or analogues of rapamycin. The PI3K/mTOR pathway plays a role in regulating adaptive immunity by targeting T cell proliferation and activation. Indeed, as indicated herein, PI3K/mTOR signaling appears to be a regulatory pathway of the innate immune host response.

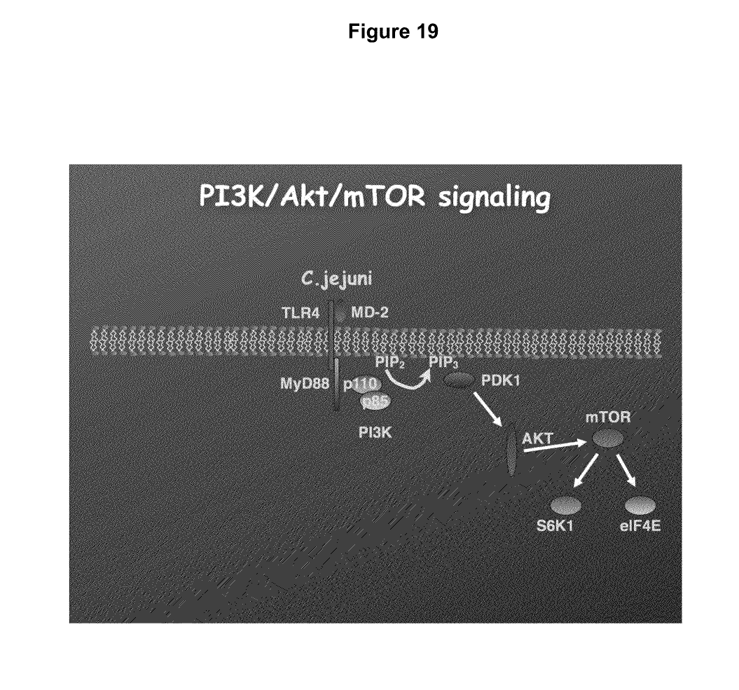

Disclosed herein for the first time is the discovery linking PI3K, mTOR and/or the PI3K/mTOR signaling pathway (see FIG. 19) with the host response to C. jejuni infection. Thus, disclosed herein are novel findings regarding the ability of PI3K and/or mTOR signaling to regulate C. jejuni induced expression of inflammatory mediators, neutrophil infiltration and bacterial clearance, as well as novel therapeutic targets for C. jejuni induced intestinal inflammation.

To elaborate, disclosed herein is the discovery that C. jejuni-induced PI3K and/or mTOR signaling leads to increased NF-κB activity and induction of NF-κB-dependent genes (cytokines/chemokines). PI3K and/or mTOR blockade with a pharmacological inhibitor strongly attenuated campylobacteriosis in II10^{-/-} mice. Moreover, rapamycin was able to treat established C. jejuni-induced intestinal inflammation. Thus, disclosed herein are methods of assessing, modulating, attenuating, reversing and treating campylobacteriosis. Also described are agents for modulating, attenuating, reversing and treating campylobacteriosis.

A close examination of human campylobacteriosis suggests a preponderant role of neutrophils in the course of the pathology. Histological assessment showed the presence of numerous crypt abscesses and neutrophil infiltration in the intestinal mucosa of infected patients. Although the main biological function of neutrophils is to ingest and eliminate invading microorganisms, excessive infiltration of these innate immune cells and release of various degradative enzymes and oxidative products cause extensive collateral tissue damage to the host. As disclosed herein, blocking mTOR with rapamycin decreased colonic II-17a and Cxcl2 expression, which correlated with reduction of neutrophils infiltration and crypt abscesses and with disease improvement. Thus, disclosed herein are methods of modulating, attenuating, reversing and treating symptoms of campylobacteriosis and infections by other enteropathogenic microorganisms. Enteropathogenic microorganisms are those microorganisms capable of causing disease in the intestinal tract. Also described are agents for modulating, attenuating, reversing and treating these symptoms.

Another function of mTOR signaling is the regulation of autophagy. The findings disclosed herein indicate that C. jejuni infiltration in the colon, MLN and spleen is dramatically reduced in rapamycin-treated mice, suggesting increased bacterial killing. Interestingly, S. typhimurium induced colitis was also inhibited by rapamycin exposure, suggesting that mTOR is a target of other enteropathogenic microorganisms. Thus, disclosed herein are methods of modulating, attenuating, reversing and treating campylobacteriosis and infections by other enteropathogenic microorganisms by modulating mTOR signaling and/or increasing bacterial killing. Also described are agents to for modulating mTOR signaling and/or increasing bacterial killing.

In some embodiments, methods are provided for modulating, inhibiting and/or blocking the PI3K/mTOR pathway (FIG. 19) and any resulting cellular/molecular events. In some embodiments, methods of modulating, inhibiting and/or blocking the PI3K/mTOR pathway pathway can treat, attenuate or reverse an enteropathogenic microorganism infection in a subject. In some embodiments, the enteropathogenic microorganism infection can comprise an infection of C. jejuni, S. typhimurium, and/or Shigella. In some embodiments, methods are provided for modulating, attenuating, reversing and/or treating campylobacteriosis. In some embodiments, a method of treating enteritis in a subject can comprise providing a subject suffering from enteritis, and administering to the subject an inhibitor or modulator of the PI3K/mTOR pathway or a component thereof, such as but not limited to PI3K, PI3Kp110γ, PI3Kp110β, PI3Kp110δ, PI3Kp110α, AKT, mTOR and/or S6K1, wherein the enteritis is treated. In some embodiments treating the enteritis comprises reduced intestinal inflammation and/or increased bacterial clearance.

In some embodiments, agents, compositions, or therapeutic compounds are provided for modulating, inhibiting and/or blocking the PI3K/mTOR pathway or a component thereof, such as but not limited to PI3K, PI3Kp110γ, PI3Kp110β, PI3Kp110δ, PI3Kp110α, AKT, mTOR and/or S6K1, of cellular/molecular events. In some embodiments, agents, compositions, or therapeutic compounds capable of modulating, inhibiting and/or blocking the PI3K/mTOR pathway can treat, attenuate or reverse an enteropathogenic microorganism infection in a subject. In some embodiments, an inhibitor of a component or components of the PI3K/mTOR pathway is provided. In some embodiments, the mTOR inhibitor is rapamycin. In some embodiments, the mTOR inhibitor is a derivative or analogue of rapamycin. In some embodiments, the inhibitor of mTOR is an agent/composition/compound available under the registered trademark RAPAMUNE®, TORISEL®, AFINITOR® or ZORTRESS®. In some embodiments, the inhibitor of PI3K is wortmannin. In some embodiments, the inhibitors of PI3Kγ are AS252424, thiazolidinones, thiazolidinones, and 2-aminothiazoles. In some embodiments, a therapeutic composition to treat enteritis in a subject is provided, the therapeutic composition comprising a PI3K/mTOR pathway inhibitor, and a pharmaceutically acceptable carrier.

Based on the disclosure herein linking the PI3K/mTOR pathway with the host response to C. jejuni infection methods, in some embodiments, methods are provided for identifying new agents, compositions, or therapeutic compounds that can modulate, inhibit and/or block the PI3K/mTOR pathway of cellular/molecular events. In some embodiments, methods are provided for identifying new agents, compositions, or therapeutic compounds for modulating, attenuating, reversing and/or treating campylobacteriosis and infections by other enteropathogenic microorganisms. Provided in some embodiments are methods of identifying an agent to treat enteritis, the method comprising providing a test sample comprising a polypeptide of the PI3K/mTOR pathway, administering a test molecule to the test sample, and determining the effect of the test molecule on the activity of the polypeptide of the PI3K/mTOR pathway. In some aspects, the polypeptide of the PI3K/mTOR pathway can comprise mTOR complex 1, mTOR complex 2, PI3Kγ, PI3Kp110γ, PI3Kp110β, PI3Kp110δ, PI3Kp110α, AKT, and/or S6K1. In some embodiments, the effect of the test molecule on the activity of the polypeptide of the PI3K/mTOR pathway can be a modulatory effect, wherein the modulatory effect is an inhibition of a signaling activity of the polypeptide of the PI3K/mTOR pathway.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including,” “containing,” or “characterized by” is inclusive or open-ended and does not exclude additional, unrecited elements or method steps. “Comprising” is a term of art used in claim language which means that the named elements are present, but other elements can be added and still form a construct or method within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p value”. Those p values that fall below a user-defined cutoff point are regarded as significant. In some embodiments, a p value less than or equal to 0.05, in some embodiments less than 0.01, in some embodiments less than 0.005, and in some embodiments less than 0.001, are regarded as significant. Accordingly, a p value greater than or equal to 0.05 is considered not significant.

As used herein, the term “subject” refers to any organism for which application of the presently disclosed subject matter would be desirable. The subject treated in the presently disclosed subject matter in its many embodiments is desirably a human subject, although it is to be understood that the principles of the presently disclosed subject matter indicate that the presently disclosed subject matter is effective with respect to all vertebrate species, including mammals, which are intended to be included in the term “subject”. Moreover, a mammal is understood to include any mammalian species in which treatment of enteritis is desirable, particularly agricultural and domestic mammalian species.

The term “modulate” can refer to a change in the activity or expression level of a gene, or a level of RNA molecule or equivalent RNA molecules encoding one or more proteins or protein subunits, or activity of one or more proteins or protein subunits that is up regulated or down regulated, such that expression, level, or activity is greater than or less than that observed in the absence of the modulator. For example, the term “modulate” can mean “inhibit” or “suppress”, but the use of the word “modulate” is not limited to this definition. By way of example and not limitation, “modulation” of the PI3K/mTOR pathway, or a component thereof, e.g. PI3K, PI3Kp110γ, PI3Kp110δ, PI3Kp110δ, PI3Kp110α, AKT, mTOR and/or S6K1, can refer to a change in the activity or expression or one or more components of the PI3K/mTOR pathway, and/or can refer to a change in one or more molecular or cellular signaling events associated with the pathway. In some embodiments, “modulation” of the PI3K/mTOR pathway can refer to a decrease or increase in one or more molecular or cellular signaling events associated with the pathway as compared to the same molecular or cellular signaling events in a normal or healthy subject, tissue or cell, or a subject, tissue or cell not exposed to the modulator. In some embodiments, a “modulation” that results in a decrease, an inhibition, an increase or any other change as compared to the standard or norm can be a difference of about 5%, 10%, 15%, 20%, 25%, 30%, 40%, 50%, 60%, 70%, 80%, 90% or more.

### Methods of Screening Compounds for the Ability to Modulate PI3K/mTOR Signaling Pathway

PI3K/mTOR pathway modulators can be identified by providing a test sample comprising a polypeptide of the PI3K/mTOR pathway (FIG. 19), administering a test molecule to the test sample, and determining the effect of the test molecule on the activity of polypeptide of the PI3K/mTOR pathway. In some embodiments, the polypeptide of the PI3K/mTOR pathway can include but is not limited to a mTOR complex, mTOR complex 1 (mTORC1), mTOR complex 2 (mTORC2) or PI3Kγ. As one of ordinary skill in the art will appreciate, any polypeptide or component of the PI3K/mTOR pathway (FIG. 19) can serve as a marker for testing the effect of a test molecule on the signaling capacity of the PI3K/mTOR pathway. By way of example and not limitation, the polypeptide of the PI3K/mTOR pathway can comprise PI3K, AKT, mTOR, and/or p70S6K, or any other component in the PI3K/mTOR pathway as illustrated in FIG. 19. A test molecule can be any molecule having any chemical structure. For example, a test molecule can be a polypeptide, carbohydrate, lipid, amino acid, nucleic acid, fatty acid, or steroid. In addition, a test molecule can be lipophilic, hydrophilic, plasma membrane permeable, or plasma membrane impermeable. In some embodiments, the test molecule is selected from the group including but not limited to a polypeptide, a nucleic acid oligonucleotide, optionally an siRNA to one or more of the isoform mRNAs of PI3K/mTOR components, an exogenous vector coding for a nucleic acid oliognucleotide or polypeptide, a carbohydrate, a lipid, an amino acid, a fatty acid, a steroid, and a low molecular weight organic molecule.

Determining the effect of the test molecule on the activity of the PI3K/mTOR pathway, or one or more components thereof, can comprise identifying a modulatory effect on the signaling activity of the PI3K/mTOR pathway or a specific component thereof. That is, a PI3K/mTOR pathway modulating compound can comprise a compound capable of modulating, and in some embodiments inhibiting or blocking, the ability of one or more components of the PI3K/mTOR pathway to effectively participate in the PI3K/mTOR pathway signaling, and/or for the PI3K/mTOR pathway as a whole to effectively signal as would occur under normal conditions. mTOR, or the mTOR complex composed of mTOR complex 1 (mTORC1) and mTOR complex 2 (mTORC2), is a downstream target of PI3Ks and is sensitive to the pharmacological inhibitor rapamycin. The PI3K/mTOR pathway has been implicated in regulating adaptive immunity by targeting T cell proliferation and activation. PI3K/mTOR signaling is also a regulatory pathway of the innate immune host response.

The presently disclosed subject matter provides several assays that can be used to identify PI3K/mTOR pathway modulators. Such assays involve monitoring at least one of the biological responses mediated by the PI3K/mTOR pathway in cells expressing one or more components of the PI3K/mTOR pathway. By way of example and not limitation, a PI3K/mTOR pathway modulator can be identified using an assay in cells transfected with a nucleic acid molecule that expresses a polypeptide having an activity of a PI3K/mTOR pathway polypeptide. By way of example and not limitation, rapamycin, or an analogue or derivative of rapamycin, is a mTOR inhibitor.

In accordance with the presently disclosed subject matter there are also provided methods for screening candidate compounds for the ability to modulate in vivo PI3K/mTOR pathway component levels and/or activities. Representative modulators of PI3K/mTOR pathway component levels and/or activities can comprise modulators of transcription or expression of PI3K/mTOR polypeptides or compounds in the PI3K/mTOR signaling pathway. Compositions that modulate (i.e. increase or decrease) the transcription or expression of PI3K/mTOR polypeptide-encoding genes have application for the modulation of the biological activity of PI3K/mTOR polypeptide.

Thus, provided herein is a method for discovery of compounds that modulate the expression levels of polypeptide-encoding genes for polypeptides involved in the PI3K/mTOR pathway. By way of example and not limitation, such genes can comprise the gene for PI3Kγ (SEQ ID NO: 14) and/or mTOR (SEQ ID NO: 15). Of course, as one of ordinary skill in the art will appreciate, the gene for any known component of the PI3K/mTOR pathway, e.g. PI3K, PI3Kp110γ, PI3Kp110β, PI3Kp110δ, PI3Kp110α, AKT, mTOR, S6K1, can be employed in this method. The general approach is to screen compound libraries for substances which increase or decrease expression of polypeptide-encoding genes for polypeptides involved in the PI3K/mTOR pathway. Exemplary techniques are described in U.S. Pat. Nos. 5,846,720 and 5,580,722, the entire contents of each of which are herein incorporated by reference.

In some embodiments, PI3K/mTOR modulating compounds that are discovered by the presently disclosed subject matter can be used to treat enteritis in a subject. In some embodiments, compounds discovered using the above methods can be used to treat enteritis in a subject, such as enteritis caused by Campylobacter jejuni, Salmonella typhimurium, Enteropathogenic Escherichia coli and Shigella, using methods of treatment as described herein.

While the following terms are believed to be well understood by one of skill in the art, the following definitions are set forth to facilitate explanation of the invention.

“Transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript. “Expression” generally refers to the cellular processes by which a biologically active polypeptide is produced from RNA.

“Transcription factor” means a cytoplasmic or nuclear protein which binds to such gene, or binds to an RNA transcript of such gene, or binds to another protein which binds to such gene or such RNA transcript or another protein which in turn binds to such gene or such RNA transcript, so as to thereby modulate expression of the gene. Such modulation can additionally be achieved by other mechanisms; the essence of “transcription factor for a gene” is that the level of transcription of the gene is altered in some way.

In accordance with the presently disclosed subject matter there is provided a method of identifying a candidate compound or molecule that is capable of modulating the transcription level of a gene encoding a PI3K/mTOR polypeptide and thus is capable of acting in the modulation of PI3K/mTOR polypeptide effects. Such modulation can be direct, i.e., through binding of a candidate molecule directly to the nucleotide sequence, whether DNA or RNA transcript, or such modulation can be achieved via one or more intermediaries, such as proteins other than PI3K/mTOR polypeptide which are affected by the candidate compound and ultimately modulate PI3K/mTOR polypeptide transcription by any mechanism, including direct binding, phosphorylation or dephosphorylation.

This method comprises contacting a cell or nucleic acid sample with a candidate compound or molecule to be tested. These samples contain nucleic acids which can contain elements that modulate transcription and/or translation of a PI3K/mTOR polypeptide gene, such as a promoter or putative upstream regulatory region (representative of such as disclosed herein), and a DNA sequence encoding a polypeptide which can be detected in some way. Thus, the polypeptide can be described as a “reporter” or “marker.” Optionally, the candidate compound directly and specifically transcriptionally modulates expression of the PI3K/mTOR polypeptide-encoding gene.

The DNA sequence is coupled to and under the control of the promoter, under conditions such that the candidate compound or molecule, if capable of acting as a transcriptional modulator of the gene encoding PI3K/mTOR polypeptide, causes the polypeptide to be expressed and so produces a detectable signal, which can be assayed quantitatively and compared to an appropriate control. Candidate compounds or molecules of interest can include those which increase or decrease, i.e., modulate, transcription from the regulatory region. The reporter gene can encode a reporter known in the art, such as luciferase, or it can encode PI3K/mTOR polypeptide.

In certain embodiments of the presently disclosed subject matter the polypeptide so produced is capable of complexing with an antibody or is capable of complexing with biotin. In this case the resulting complexes can be detected by methods known in the art. The detectable signal of this assay can also be provided by messenger RNA produced by transcription of said reporter gene. Exactly how the signal is produced and detected can vary and is not the subject of the presently disclosed subject matter; rather, the presently disclosed subject matter provides the nucleotide sequences and/or putative regulatory regions of a PI3K/mTOR polypeptide for use in such an assay. The molecule to be tested in these methods can be a purified molecule, a homogenous sample, or a mixture of molecules or compounds. Further, in representative embodiments, the DNA in the cell can comprise more than one modulatable transcriptional regulatory sequence.

In accordance with the presently disclosed subject matter there is also provided a rapid and high throughput screening method that relies on the methods described above. This screening method comprises separately contacting each of a plurality of substantially identical samples. In such a screening method the plurality of samples preferably comprises more than about 10⁴ samples, or more preferably comprises more than about 5×10⁴ samples.

Method of Modulating the Biological Activity of PI3K/mTOR Pathway

Also disclosed herein are methods of modulating the PI3K/mTOR pathway, and particularly a polypeptide or component of the PI3K/mTOR pathway, in a subject or a biological sample. By way of example and not limitation, a polypeptide or component of the PI3K/mTOR pathway (FIG. 19) can comprise a mTOR complex, and/or one or more of the two entities of a mTOR complex: mTOR complex 1 (mTORC1) and mTOR complex 2 (mTORC2), PI3Kγ, PI3Kp110γ, PI3Kp110β, PI3Kp110δ, PI3Kp110α, AKT, mTOR and/or S6K1. As would be appreciated by one of ordinary skill in the art, the disclosed methods of modulating the PI3K/mTOR signaling pathway can comprise modulating any one or more of the known constituents in the PI3K/mTOR pathway in a subject or biological sample. In some embodiments, the method comprises contacting the biological sample and/or subject with an agent for modulating expression, activity or both expression and activity of a constituent in the PI3K/mTOR pathway. In some embodiments, a biological sample can be a tissue, organ or site present in a subject. In some embodiments, the subject can be a human subject. In some embodiments, the subject can be a subject infected with or believed to be infected with an enteropathogenic microorganism. In some embodiments, the enteropathogenic microorganism infection can comprise an infection of C. jejuni, S. typhimurium, Enteropathogenic Escherichia coli or Shigella. In some embodiments, the subject can be suffering from enteritis.

In some embodiments the agent for modulating expression, activity or both expression and activity of a constituent in the PI3K/mTOR pathway is selected from the group including but not limited to a polypeptide, a nucleic acid oligonucleotide, optionally an siRNA to one or more isoform mRNAs of a constituent in the PI3K/mTOR pathway, a vector coding for a nucleic acid oligonucleotide or polypeptide, a carbohydrate, a lipid, an amino acid, a fatty acid, a steroid, and a low molecular weight organic molecule.

In some embodiments, the presently disclosed subject matter takes advantage of RNAi technology (for example shRNA, siRNA and miRNA molecules and ribozymes) to cause the down regulation of cellular genes, a process referred to as RNA interference (RNAi). As used herein, “RNA interference” (RNAi) refers to a process of sequence-specific post-transcriptional gene silencing mediated by a small interfering RNA (siRNA) or short hairpin RNA (shRNA) molecules, miRNA molecules or synthetic hammerhead ribozymes. See generally Fire et al. (1998) and U.S. Pat. No. 6,506,559. The process of RNA interference (RNAi) mediated post-transcriptional gene silencing is thought to be an evolutionarily conserved cellular defense mechanism that has evolved to prevent the expression of foreign genes (Fire, 1999).

As used herein, the terms “inhibit”, “suppress”, “down regulate”, “knock down”, and grammatical variants thereof are used interchangeably and refer to an activity whereby gene expression or a level of an RNA encoding one or more gene products is reduced below that observed in the absence of a composition of the presently disclosed subject matter.

With respect to the therapeutic methods of the presently disclosed subject matter, a representative subject is a vertebrate subject. A representative example of a vertebrate is a warm-blooded vertebrate. A representative example of a warm-blooded vertebrate is a mammal. A representative example of a mammal is a human. Additionally, as used herein and in the claims, the term “patient” can include both human and animal patients, and thus, veterinary therapeutic uses are provided in accordance with the presently disclosed subject matter.

Provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

A “therapeutic composition” or a “pharmaceutical composition” as described herein optionally but typically comprises a composition that includes a pharmaceutically acceptable carrier. In some embodiments, the presently disclosed subject matter provides pharmaceutical compositions comprising a polypeptide, polynucleotide or PI3K/mTOR inhibitor of the presently disclosed subject matter and a physiologically acceptable carrier. In some embodiments, constructs are conjugated to a carrier, for example a nanoparticle or an antibody to direct its delivery to the target cells. The carrier (e.g. nanoparticle) conjugated to the agent can be injected in an acceptable pharmaceutical diluent. In some embodiments, an agent is delivered to a target cell by a delivery vehicle, such as but not limited to a viral vector, an antibody, an aptamer, or a nanoparticle.

A composition of the presently disclosed subject matter is typically administered parenterally in dosage unit formulations containing standard, well-known nontoxic physiologically acceptable carriers, adjuvants, and vehicles as desired. The term “parenteral” as used herein includes intravenous, intra-muscular, intra-arterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Preferred carriers include neutral saline solutions buffered with phosphate, lactate, Tris, and the like. Of course, one purifies the vector sufficiently to render it essentially free of undesirable contaminants, such as defective interfering adenovirus particles or endotoxins and other pyrogens such that it does not cause any untoward reactions in the individual receiving the vector construct. A preferred means of purifying the vector involves the use of buoyant density gradients, such as cesium chloride gradient centrifugation.

A transfected cell can also serve as a carrier. By way of example, a cell can be removed from an organism, transfected with a polynucleotide of the presently disclosed subject matter using methods set forth above and then the transfected cell returned to the organism (e.g. injected intra-vascularly).

### EXAMPLES

The following Examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-4

Mice

All animal protocols were approved by the Institutional Animal Care and Use Committee of the University of North Carolina at Chapel Hill (Chapel Hill, N.C., United States of America). Germ-free 8 to 12 week old II10^{-/-}; NF-κBEGFP (129/SvEv; C57BL/6 mixed background) mice were transferred from germ-free isolators and immediately gavaged with 10⁹ C. jejuni cfu/mouse (strain 81-176 (Korlath et al., 1985)). Mice were then housed under Specific Pathogen-Free (SPF) conditions and sacrificed at 5 or 12 days. Germ-free II10^{-/-}; NF-κB^{EGFP} mice were also gavaged with 10⁷ Salmonella typhimurium (S. typhimurium strain CA32) cfu/mouse for 2 days or 10⁷ Escherichia coli (E. coli strain NC 101) cfu/mouse for 12 days. Conventionally-derived II10^{-/-}; NF-κB^{EGFP} mice were put on an antibiotic cocktail (streptomycin 2 g/L, gentamycin 0.5 g/L, bacteriocin 1 g/L and ciprofloxacin 0.125 g/L) in water for 7 days and antibiotics withdrawn 24 hours prior to infection. The mice were then gavaged with 10⁹ C. jejuni cfu/mouse for 14 days. To inhibit mTOR signaling, mice were injected daily intraperitoneally (i.p.) with rapamycin (1.5 mg/kg) (Fisher Scientific, Hampton, N.H., United States of America). At the end of the experiment, mice were euthanized using CO₂ intoxication. Colon, mesenteric lymph nodes (MLN) and spleen were collected for further processing RNA, protein or C. jejuni culture on Campylobacter selective blood plates (Remel, Thermo Scientific, Hampton, N.H., United States of America) for 48 h at 37° C. using the GasPak system (Becton Dickinson, Franklin Lakes, N.J., United States of America). C. jejuni treated with up to 1 mM rapamycin in PBS were cultured on the plates and showed normal growth. Colons were fixed in 10% buffered formalin (Fisher Scientific, Hampton, N.H., United States of America) overnight, paraffin-embedded, sectioned, and stained with H&E for histological evaluation. Images were acquired using a DP71 camera and DP Controller 3.1.1.276 (Olympus, Center Valley, Pa., United States of America). Intestinal inflammation was scored by evaluating the degree of lamina propria immune cell infiltration, goblet cell depletion, architectural distortion, as well as crypt hyperplasia, ulceration, and abscesses using a score from 0-4. (Lippert et al., 2009). Cecal and colonic inflammation induced by S. typhimurium was evaluated using a score from 0-13 as described. (Hapfelmeier et al., 2005).

CD4+ Cell Depletion

C. jejuni infected mice were injected with anti-CD4 antibody (BioXcell, West Lebanon, N.H., United States of America) (i.p. 0.5 mg/mouse, every 3 days) to deplete CD4+ cells. At the end of the experiment, mice were euthanized using CO₂ intoxication. Colons were resected and processed for H&E staining and colitis evaluation. Spleen and MLN were collected and processed into single cell suspensions after erythrocyte lysis.

Western Blotting

Whole tissues were lysed in Laemmli buffer and 20 μg of protein was separated by SDS-PAGE, transferred to nitrocellulose membranes and protein detected using enhanced chemiluminescence reaction (ECL) as described previously. (Lippert et al., 2009). Primary antibodies used were phospho-p70S6K (T389) and p70S6K (Cell Signaling, Danvers, Mass., United States of America), EGFP (Sigma, St. Louis, Mo., United States of America), LC3 I/II (MBL) and Actin.

Enhanced GFP (EGFP) Macro- and Micro-Imaging

II10^{-/-}; NF-κB^{EGFP} mice were sacrificed; the colon was dissected and immediately imaged using a charge-coupled device camera in a light-tight imaging box with a dual-filtered light source and emission filters specific for EGFP (LT-99D2 Illumatools; Lightools Research, Encinitas, Calif., United States of America). For microimaging, colonic segments were cut open and fixed in 4% paraformaldehyde overnight at 4° C., and then permeabilized in 2% triton overnight at 4° C. The tissues were then immersed in FocusClear (CelExplorer labs) and then stained with DAPI. The tissues were put on slides with lumen side facing the lens. NF-κB derived EGPF were examined using OLYMPUS® FV1000 confocal multiphoton upright microscope system with OLYMPUS® Fluoview 2.0 software (Olympus, Center Valley, Pa., United States of America). Acquired images were reconstructed into 3-D using Imaris (Bitplane Inc., South Windsor, Conn., United States of America).

Fluorescence In Situ Hybridization (FISH)

Cy3-tagged 5′AGCTAACCACACCTTATACCG3′ (SEQ ID NO: 1; Poppert et al., 2008) was used to probe the presence of C. jejuni. Deparaffinized, formalin-fixed 5 μm thick sections were incubated for 15 minutes in lysozyme (300,000 Units/ml lysozyme; Sigma-Aldrich, St. Louis, Mo., United States of America) buffer (25 mM Tris pH 7.5, 10 mM EDTA, 585 mM sucrose, and 0.3 mg/ml sodium taurocholate) at room temperature and hybridized overnight at 46° C. in hybridization chambers with the oligonucleotide probe (final concentration of 5 ng/μl in a solution of 30 percent formamide, 0.9 M sodium chloride, 20 mM Tris pH 7.5, and 0.01% sodium dodecyl sulfate). Tissue sections were washed for 20 minutes at 48° C. in washing buffer (0.9 M sodium chloride, 20 mM Tris pH 7.5) and once in distilled water for 10 seconds, dried at 46° C., mounted with DAPI mount media, and imaged using a Zeiss (Carl Zeiss, Thornwood, N.Y., United States of America) LSM710 Spectral Confocal Laser Scanning Microscope system with ZEN 2008 software. Acquired images were analyzed using BioimageXD. (Kankaanpaa et al., 2006). For whole tissue FISH, colons were fixed in 4% paraformaldehyde overnight at 4° C., 2% triton overnight at 4° C., and C. jejuni probe was added and incubated overnight at 46° C. The whole tissue was immersed in FOCUSCLEAR™, imaged on an OLYMPUS® FV1000 microscope and reconstructed into 3-D using Imaris.

Immunohistochemistry (IHC)

Deparaffinized colon sections were treated with 3% H₂O₂ for 10 minutes to quench endogenous peroxidases. Antigen retrieval was performed using microwave heating for 10 min in 10 mM citrate buffer pH 6.0. Sections were blocked in 5% goat serum TBS-T for 1 hour. The slides were incubated overnight at 4° C. with an anti-MPO antibody (1:400) (Thermo Scientific, Hampton, N.H., United States of America). Secondary biotinylated antibody was diluted at 1:1000 with the VECTASTAIN® ABC Elite Kit (Vector Laboratories, Inc., Burlingame, Calif., United States of America). Visualization was performed using DAB chromogen (Dako, Inc., Carpinteria, Calif., United States of America). Finally, sections were counterstained with hematoxylin for 5 seconds. Sections were imaged at the OLYMPUS® microscope using DP 71 camera and DP Controller 3.1.1.276 (Olympus, Center Valley, Pa., United States of America).

Real-Time Reverse-Transcription Polymerase Chain Reaction

Total RNA from colonic tissue was extracted using TRIzol® kit (Invitrogen, Carlsbad, Calif., United States of America). Complementary DNA was prepared using M-MLV (Invitrogen). Messenger RNA levels of proinflammatory genes were determined using SYBR® Green PCR Master Mix (Applied Biosystems, Carlsbad, Calif., United States of America) on an ABI 7900HT Fast Real-Time PCR System and normalized to Gapdh. The polymerase chain reactions were performed according to the manufacturer's recommendation. The following gene primers were used: Gapdh_forward: 5′-GGTGAAGGTCGGAGTCAACGGA-3′ (SEQ ID NO: 2), Gapdh_reverse: 5′-GAGGGATCTCGCTCCTGGAAGA-3′ (SEQ ID NO: 3), II-1_forward: 5′-GCCCATCCTCTGTGACTCAT-3′ (SEQ ID NO: 4), II-1_reverse: 5′-AGGCCACAGGTATTTTGTCG-3′ (SEQ ID NO: 5), Cxcl2_forward: 5′-AAGTTTGCCTTGACCCTGAA-3′ (SEQ ID NO: 6), Cxcl2_reverse: 5′-AGGCACATCAGGTACGATCC-3′ (SEQ ID NO: 7), II-17a_forward: 5′-TCCAGAAGGCCCTCAGACTA-3′ (SEQ ID NO: 8), II-17a_reverse: 5′-ACACCCACCAGCATCTTCTC-3′ (SEQ ID NO: 9), II12p40_forward: GAAGTTCAACATCAAGAGCAGTAG (SEQ ID NO: 10), II12p40_reverse: AGGGAGAAGTAGGAATGGGG (SEQ ID NO: 11), Tnf forward: ATGAGCACAGAAAGCATGATC (SEQ ID NO: 12), Tnf reverse: TACAGGCTTGTCACTCGAATT (SEQ ID NO: 13).

Transmission Electron Microscopy (TEM)

Colon segments were fixed in 2% paraformaldehyde/2.5% glutaraldehyde in 0.15 mol/L sodium phosphate buffer, pH 7.4, and stored at 4° C. for several days before processing. Following several washes in 0.15 mol/L sodium phosphate buffer, the samples were postfixed in 1% osmium tetroxide/1.25% potassium ferrocyanide in 0.15 mol/L sodium phosphate buffer, pH 7.4, for 1 hour. Samples were dehydrated in a graded series of ethanols, followed by propylene oxide, and infiltrated and embedded in POLY/BED® 812 resin (Polysciences, Inc., Warrington, Pa., United States of America). One-micrometer semithin transverse sections were cut with a glass knife, mounted on slides, stained with 1% toluidine blue, and viewed using a light microscope to select the region of interest for TEM. Ultrathin sections (70 nm) were cut using a diamond knife, mounted on 200 mesh copper grids, and stained with 4% aqueous uranyl acetate and Reynold's lead citrate. Samples were observed using a LEO EM910 transmission electron microscope operating at 80 kV (Carl Zeiss, Thornwood, N.Y., United States of America), and digital images were acquired using a Gatan ORIUS® SC1000 CCD Digital Camera with Digital Micrograph 3.11.0 (Gatan, Inc., Pleasanton, Calif., United States of America).

Flow Cytometry Analysis

To assess blood neutrophils, mice were sacrificed and peripheral blood was drawn and mixed with an anticoagulant (20 μL 96 mmol/L EDTA). White blood cells were collected following erythrocyte lysis. The cells were then incubated with antibodies against CD45 (PE conjugated), Gr-1 (fluorescein isothiocyanate conjugated) (eBiosciences, San Diego, Calif., United States of America). To analyze the change of CD4⁺ cells after antibody-mediated depletion, freshly isolated splenocytes and MLNs from C jejuni-infected mice were then incubated with PE-conjugated antibodies against CD4 (eBiosciences, San Diego, Calif., United States of America). The cells were analyzed on a Cyan flow cytometer (Beckman Coulter, Inc., Brea, Calif., United States of America) to determine the proportion of total immune cells, neutrophils, or CD4⁺ T cells, respectively. The results were then analyzed on Summit software (Beckman Coulter, Inc., Brea, Calif., United States of America).

Enzyme-Linked Immunosorbent Assay

Colonic tissue, MLNs, and spleen were weighed, minced with scissors, and incubated in 1 mL 3% fetal bovine serum and 1% antibiotics RPMI medium in a 24-well plate. After 18-hour incubation, medium was collected and centrifuged at 7000 rpm for 5 minutes. Supernatant was collected and stored at −80° C. IL-1β and IL-17 (A) concentrations were assessed by enzyme-linked immunosorbent assay kits (R&D Systems, Minneapolis, Minn., United States of America) following the manufacturer's specification. The IL-17 enzyme-linked immunosorbent assay has no cross-reactivity to IL-17B, C, D, E, and F.

C jejuni Killing Assay Using Primary Splenocytes

Mice (8 to 12 weeks of age; II10^{-/-}) were sacrificed, and spleens were resected and homogenized using frosted glass slides in RPMI 1640 medium supplemented with 2% fetal bovine serum, 2 mmol/L L-glutamine, and 50 umol/L 2-mercaptoethanol. After centrifugation at 1500 rpm for 5 minutes, red blood cells were lysed in 5 mL red blood cell lysis buffer (0.82% NH₄Cl) for 3 minutes and then 10 mL RPMI medium was added and cells were filtered through a 70-μm strainer. After centrifugation at 1500 rpm for 5 minutes, pelleted cells were resuspended in 1 mL RPMI medium and counted. Splenocytes (2×10⁶ cells/well) were then plated on 6-well plates and rapamycin was added at 100 nmol/L for 45 minutes. C jejuni (multiplicity of infection, 50) was added into the plate wells in triplicate and plates were centrifuged at 2000 rpm for 15 minutes. Cells were incubated for 4 hours and washed 3 times with phosphate-buffered saline, and then 1 mL of fresh RPMI medium containing 100 ug/mL gentamycin and 100 nmol/L rapamycin was added for another hour. For O-hour time point sample collection, cells were washed 3 times before lysed in 0.1% Triton X-100. For 4-hour time point sample collection, cells were changed to RPMI 1640 medium with 10 ug/mL gentamycin and 100 nmol/L rapamycin for another 4 hours. Samples were then collected and lysed as described previously. The lysate was plated on Remel plates, and bacteria were enumerated using serial dilution.

Statistical Analysis.

Values are shown as mean±SEM as indicated. Differences between groups were evaluated with the nonparametric Mann-Whitney U test. Experiments were considered statistically significant if P values<0.05. All calculations were performed using Prim 5.0 software.

### Example 1

### mTOR Mediates C. jejuni-Induced Colitis

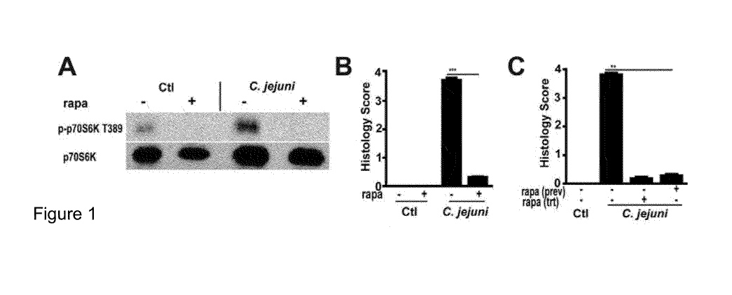

Although the role of mTOR has moved beyond that of a modulator of T cell function, the involvement of this multifunctional kinase in host responses to pathogenic bacteria has not been clearly explored. To investigate the impact of mTOR on C. jejuni-induced colitis in vivo, germ-free II10^{-/-}; NF-κB^{EGFP} mice were transferred to specific pathogen free (SPF) housing and immediately gavaged with C. jejuni (10⁹ CFU/mouse) and then injected i.p. daily with either vehicle (5% DMSO PBS) or rapamycin (1.5 mg/kg body weight) for 12 days. Western blot analysis showed that rapamycin attenuated C. jejuni induced phosphorylation of p70S6 kinase (T389), a downstream target of mTOR (FIG. 1A), as previously reported, (Lippert et al., 2009). II10^{-/-}; NF-κB^{EGFP} mice infected with C. jejuni showed severe intestinal inflammation as seen by extensive immune cell infiltration, epithelial ulceration, goblet cell depletion and crypt hyperplasia and abscesses compared to uninfected mice (FIG. 1B). Interestingly, rapamycin blocked C. jejuni-induced intestinal inflammation in II10^{-/-}; NF-κB^{EGFP} mice. Since germ free mice have an immature immune system, conventionally-derived II10^{-/-}; NF-κB^{EGFP} mice were then treated with antibiotic and then infected with C. jejuni for 12 days. As observed in germ free mice, C. jejuni induced severe intestinal inflammation in conventionally-derived II10^{-/-}; NF-κB^{EGFP} mice, an affect attenuated by rapamycin exposure (FIG. 8A).

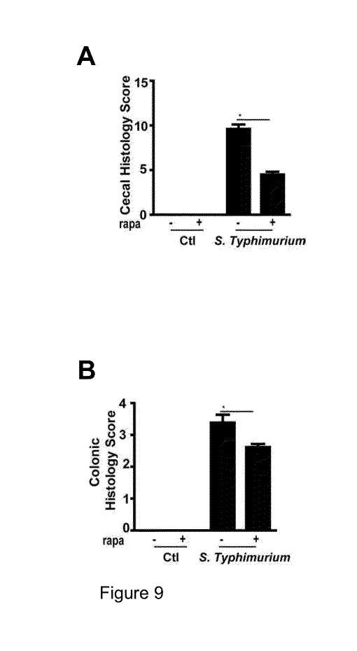

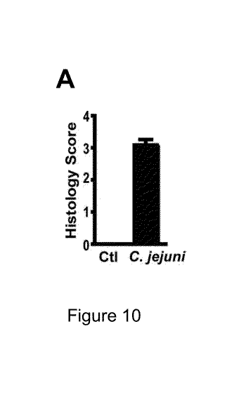

To determine the specificity of rapamycin on other colitogenic microorganisms, germ free II10^{-/-}; NF-κB^{EGFP} mice were infected with the enteric pathogen Salmonella typhimurium (10⁷ CFU/mouse) for 2 days or Escherichia coli NC 101 (10⁷ CFU/mouse) for 12 days. Interestingly, E. coli infected II10^{-/-}; NF-κB^{EGFP} mice did not develop intestinal inflammation (FIG. 9). However, II10^{-/-}; NF-κB^{EGFP} mice infected with S. typhimurium developed severe cecal and colonic inflammation, which were strongly attenuated by rapamycin exposure (FIGS. 10A-10D). These results indicate that mTOR signaling mediates deleterious responses from different enteropathogenic microorganisms.

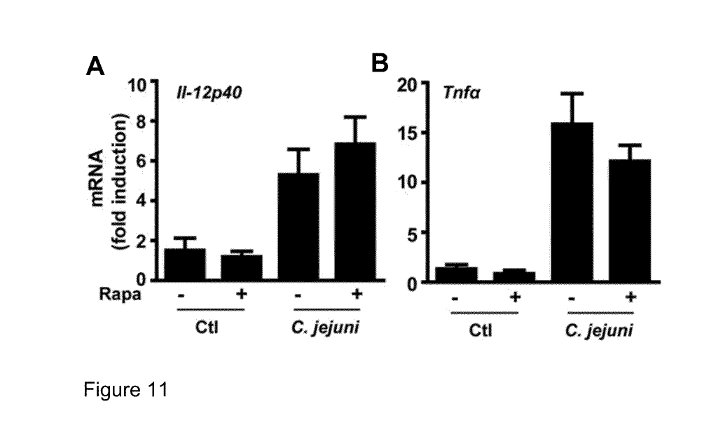

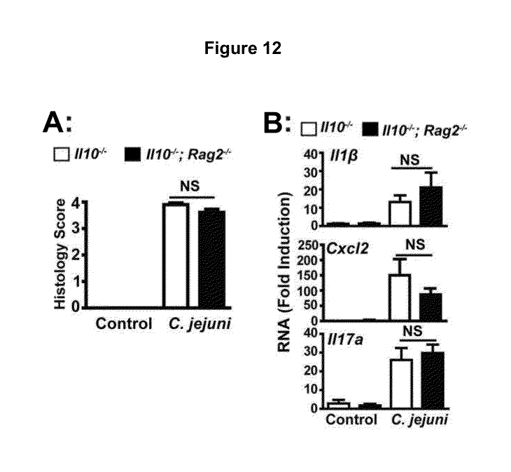

C. jejuni induced intestinal inflammation is evident as early as day 5 in II10^{-/-}; NF-κB^{EGFP} infected mice (FIGS. 11A and 11B). To further test if rapamycin may be used as an intervention agent, the inhibitor was injected daily to C. jejuni infected (4 days)II10^{-/-}; NF-κB^{EGFP} mice, and intestinal tissues were collected at 12 days. Remarkably, rapamycin treatment strongly reversed C. jejuni induced intestinal inflammation and bloody diarrhea (FIGS. 1C, 1E, 12A, and 12B).

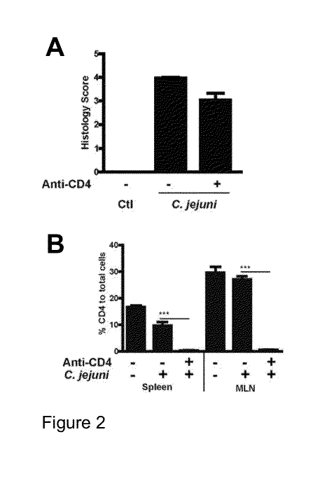

Since rapamycin has immunomodulatory effects on T cells, CD4+ T cells were depleted using an anti-CD4 antibody (0.5 mg/mouse/every three days) to address the function of these cells in the acute phase of campylobacteriosis. Notably, C. jejuni induced colitis was only slightly inhibited (−20%) in CD4+ T cell-depleted mice compared to control (FIGS. 2A-2B). Complete depletion of CD4 cells was observed in the spleen and MLN cellular compartment of antibody-treated mice (FIGS. 2C-2D). These results indicate that the early phase of C. jejuni-induced colitis is mostly mediated by an innate immune response and that rapamycin targets activities of innate immune cells.

### Example 2

### mTOR Regulates C. jejuni Induced NF-κB Activity and Proinflammatory Gene Expression

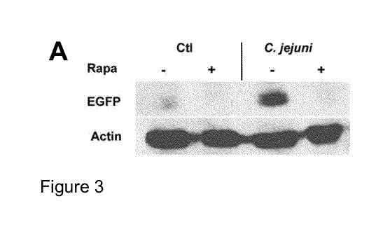

To evaluate the impact of mTOR signaling on NF-κB activity, EGFP expression in the colon of II10^{-/-}; NF-κB^{EGFP} mice was visualized using a CCD camera macroimaging system. II10^{-/-}; NF-κB^{EGFP} mice infected with C. jejuni displayed enhanced colonic NF-κB^{EGFP} expression with strongly positive lymphoid aggregates (arrow heads) compared to uninfected mice (FIG. 3A). Interestingly, the colon of rapamycin-treated, C. jejuni-infected II10^{-/-}; NF-κB^{EGFP} mice displayed reduced EGFP expression compared to untreated, infected-mice. Consistent with these findings, western blot analysis of colonic lysates demonstrated reduced EGFP expression in rapamycin-treated, C. jejuni-infected mice (FIG. 3B).

To further evaluate the distribution of NF-κB^{EGFP} positive cells within the colon, whole colonic tissue was visualized using confocal microscopy coupled with 3-D image reconstruction. In accordance with the macroimaging and Western blot data, C. jejuni-infected II10^{-/-}; NF-κB^{EGFP} mice showed numerous EGFP positive cells in the colonic lamina propria compared to uninfected mice (FIG. 3C). Strikingly, colonic EGFP tissue expression was strongly reduced in rapamycin-treated, C. jejuni-infected II10^{-/-}; NF-κB^{EGFP} mice compared to untreated mice.

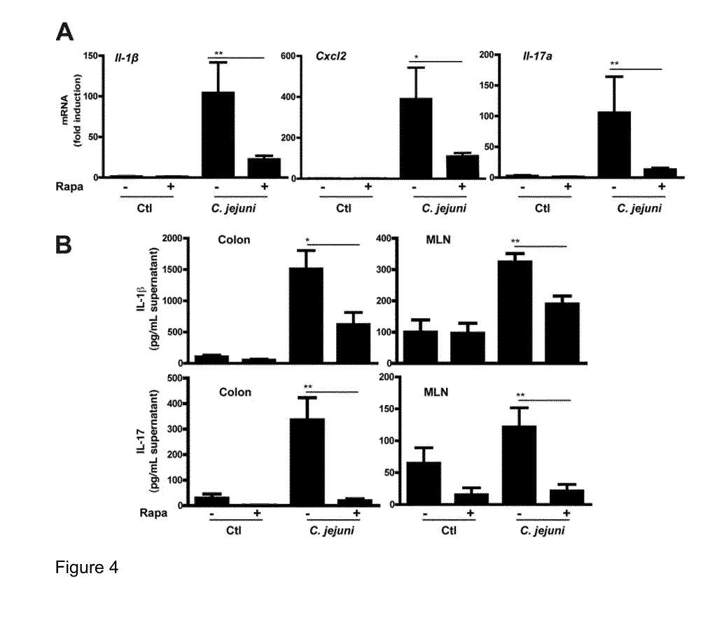

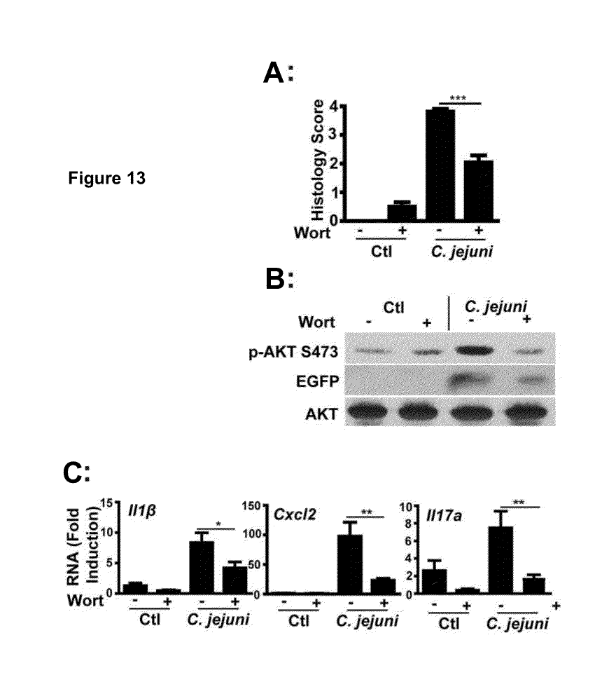

Next, the impact of rapamycin on expression of various NF-κB dependent proinflammatory mediators involved in bacterial host responses was examined. C. jejuni infection strongly induced I1-1β, Cxcl2 and II-17a mRNA accumulation in II10^{-/-}; NF-κB^{EGFP} mice, effects attenuated by 79, 75, and 92%, respectively in rapamycin-treated, C. jejuni-infected mice (FIG. 4A). Consistent with these findings, secreted IL-18 and IL-17 from supernatant of cultured colon explants was reduced by 59% and 94% respectively while production of these cytokines was attenuated by 41% and 82% in MLN of rapamycin-treated, C. jejuni-infected mice compared to control mice (FIG. 4B). Interestingly, C. jejuni-induced colonic II-12p40 and Tnfα mRNA accumulation were not significantly inhibited by rapamycin treatment, suggesting that inflammatory mediators are selectively affected by mTOR signaling (FIG. 13).

### Example 3

### Neutrophils Participate in C. jejuni-Induced Colitis

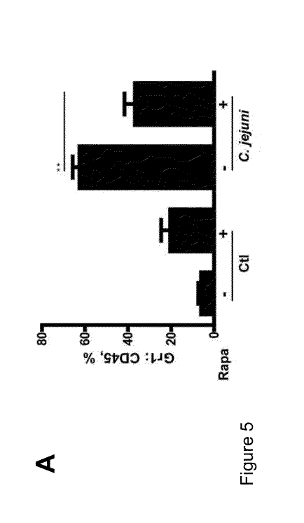

Neutrophil infiltration is a hallmark of acute infection and represents an important feature of human campylobacteriosis. (van Spreeuwel et al., 1985). As presented herein for the first time, rapamycin strongly reduced C. jejuni induced expression of the neutrophil marker myeloperoxidase (MPO) in infected II10^{-/-}; NF-κB^{EGFP} mice (FIG. 5A). Moreover, the ratio of Gr-1⁺/CD45⁺ cells significantly decreased (41%) in the blood of rapacymin-treated, C. jejuni infected mice compared to untreated, infected mice (FIG. 5B). Finally, transmission electron microscope (TEM) analysis showed neutrophil accumulation in colonic crypt, which associates with microvilli/glycocalyx destruction (FIG. 5C). Notably, these pathological features were not observed in rapamycin-treated mice (FIG. 5C, right panel).

### Example 4

### mTOR Mediates C. jejuni Invasion

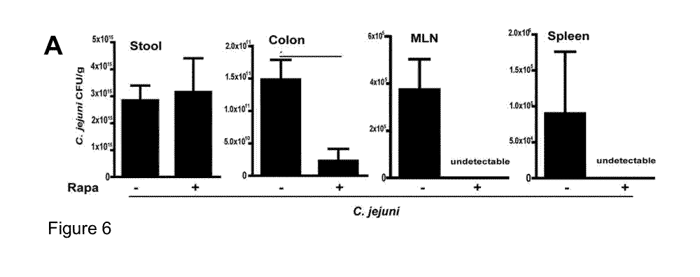

Next, the impact of mTOR signaling on intestinal and extra-intestinal C. jejuni tissue distribution was investigated. Following infection and treatment with rapamycin, C. jejuni DNA was visualized in the colon of II10^{-/-}; NF-κB^{EGFP} mice using fluorescence in situ hybridization (FISH) and confocal microscopy imaging. Interestingly, while C. jejuni was detected deeply in the inflamed crypts and in the lamina propria section of the intestine of untreated mice, C. jejuni DNA was barely detectable in rapamycin-treated mice (FIG. 6A).

To gain a better perspective of C. jejuni invasion, FISH was performed using fresh-fixed colonic tissues sections, and then imaged with confocal microscopy coupled with 3-D visualization. Interestingly, C. jejuni extensively invaded the colon of untreated mice whereas its presence is strongly reduced in rapamycin-treated mice (FIG. 6B and supplementary). To further characterize C. jejuni invasion, colonic tissue was also imaged using TEM. The presence of spirally shaped C. jejuni (arrow) lacking its outer membrane was detected in epithelial cells (FIG. 6C left panel). Also the absence of a vesicular membrane around the invading bacteria suggests that phagosome formation in epithelial cells is not a host response to the bacterium. Consistent with FISH results, intracellular C. jejuni were virtually absent and microvilli were intact in epithelial cells of rapamycin-treated mice (FIG. 6C right panel).

Reduced C. jejuni in the colon of rapamycin-treated mice suggests that the bacteria were eliminated or were able to evade/translocate to extra-intestinal tissues. To resolve this issue, samples from the stool, colon, spleen and MLN and enumerated C. jejuni on Remel Campylobacter selective plates were aseptically collected. Consistent with the FISH and TEM results, rapamycin treatment reduced C. jejuni colonic invasion by 90% compared to C. jejuni-infected, untreated mice (FIG. 6D). Furthermore, rapamycin treatment strongly reduced C. jejuni presence in the MLN and spleen of infected mice compared to untreated, infected mice. Altogether, these findings identified mTOR signaling and neutrophil infiltration as important events leading to C. jejuni invasion and pathogenesis.

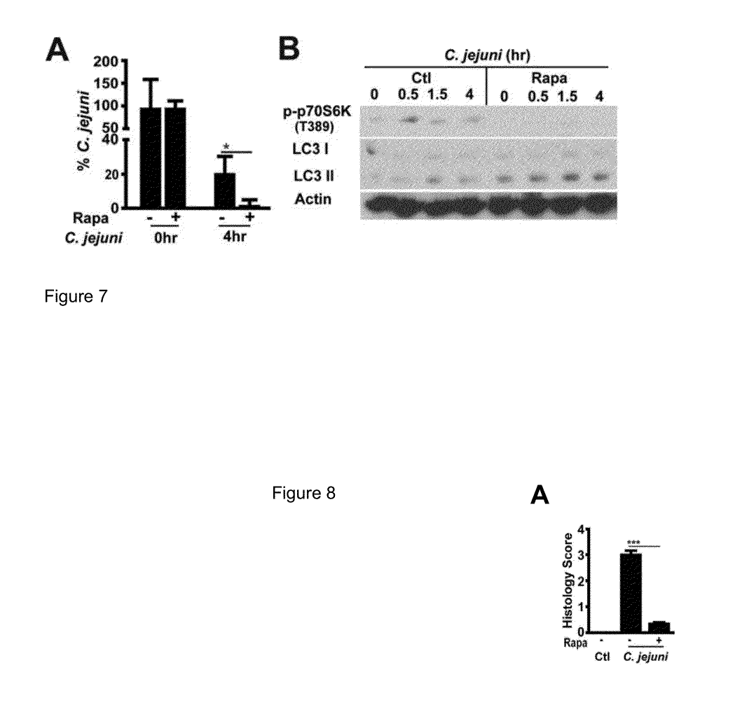

The above findings indicate that rapamycin favors bacterial clearance, which leads to decreased innate response and intestinal inflammation. To directly test the impact of rapamycin on bacterial survival, splenocytes isolated from II10^{-/-} mice were infected with C. jejuni and a gentamycin killing assay was performed. Interestingly, rapamycin enhanced C. jejuni killing in splenocytes at 4 hr compared to untreated cell (FIG. 7A). Western blot analysis showed that rapamycin attenuated C. jejuni induced phosphorylation of p70S6 kinase (T389) and enhanced LC3 II conversion, a marker of autophagy (FIG. 7B). Altogether, these findings showed that increased mTOR signaling following C. jejuni infection mediates proinflammatory response, likely by preventing host-mediated bacterial killing.

### Discussion of Examples 1-4

The fundamental molecular host response to C. jejuni infection remains virtually unknown. The data presented in Examples 1-4 demonstrates for the first time that C. jejuni-mediated intestinal inflammation is caused by activation of the host mTOR signaling pathway and neutrophil infiltration. Comparable intestinal inflammation between CD4+ cell depleted and untreated mice indicates that C. jejuni induced colitis is mostly driven by innate immune cells at day 12. Although CD4+ T cells are not involved in the development of intestinal inflammation by day 12, adaptive host response is an important hallmark of campylobacteriosis (Yuki et al., 2004). Interestingly, and in contrast to C. jejuni, the adherent invasive E. coli NC101 failed to induce colitis after 12 days, whereas severe colitis developed after 10-20 weeks of colonization (Kim et al., 2005). In addition, colitis was observed as early as two days in S. typhimurium infected II10^{-/-} mice. These findings suggest a differential ability of bacteria to trigger intestinal inflammation in II10^{-/-} mice. Using this robust and tractable animal model, it was demonstrated that C. jejuni-induced mTOR signaling leads to increased NF-κB activity and induction of NF-κB-dependent genes (cytokines/chemokines). FISH assay and electron microscopy analysis showed that C. jejuni rapidly invades the intestinal mucosal layer and triggers a host response involving neutrophil recruitment and formation of crypt ulcers. Disruption of the crypt architecture by massive neutrophil influx compromises the intestinal barrier integrity, which further favors bacterial uptake/invasion and dissemination to extra-intestinal tissues as seen by enhanced bacterial counts in the spleen and MLN. Blocking mTOR signaling with a pharmacological inhibitor such as rapamycin strongly attenuated campylobacteriosis in II10^{-/-} mice. Moreover, rapamycin was able to treat established C. jejuni-induced intestinal inflammation.

A close examination of human campylobacteriosis suggests a preponderant role of neutrophils in the course of the pathology. Histological assessment showed the presence of numerous crypt abscesses and neutrophil infiltration in the intestinal mucosa of infected patients (van Spreeuwel et al., 1985). However, up until now the functional importance of neutrophil infiltration in C. jejuni-induced pathogenesis has not been investigated. Although the main biological function of neutrophils is to ingest and eliminate invading microorganisms, excessive infiltration of these innate immune cells and release of various degradative enzymes and oxidative products cause extensive collateral tissue damage to the host. For example, neutrophil accumulation causes elevated IL-13 and CXCL2 production and subsequent joint inflammation, an effect attenuated with anti-IL13 antibody treatment (Chou et al., 2010). Interestingly, TEM showed that the microvilli and associated glycocalyx of epithelial cells in neutrophil-infiltrated crypts are virtually ablated in C. jejuni-infected mice.

The molecular events leading to neutrophil infiltration in the intestine of C. jejuni-infected mice is not entirely clear. Induction of II-17a and Cxcl2 genes is important for neutrophil expansion and recruitment (Sieve et al, 2009; Ye et al., 2001). Interestingly, blocking mTOR with rapamycin decreased colonic II-17a and Cxcl2 expression, which correlated with reduction of neutrophils infiltration and crypt abscesses and with disease improvement. Although IL-17 is a signature cytokine of the Th17 cells, the disclosed CD4 depletion experiment suggests that adaptive CD4⁺ T cells play a minor role in campylobacteriosis. IL-17 is produced by a wide array of innate immune cells including γδ1T cells, natural killer T cells and lymphoid tissue inducer cells (Xu et al., 2010). Since γδ1T cells are highly abundant in the intestinal mucosa, it is possible that these cells are the source of IL-17 in this model. Interestingly, in an E. coli infectious model, neutrophil recruitment to the peritoneal cavity is dependent on innate γδT cell-mediated IL-17 secretion (Shibata et al., 2007). Similarly, Klebsiella pneumonia-induced lung inflammation is attenuated in II-17r^{-/-} mice, which correlated with neutrophil recruitment and G-SCF and CXCL2 expression (Ye et al., 2001).

Another important function of mTOR signaling is the regulation of autophagy. The findings disclosed herein indicate that C. jejuni infiltration in the colon, MLN and spleen is dramatically reduced in rapamycin-treated mice, suggesting increased bacterial killing. Using primary splenocytes, it was discovered that rapamycin enhanced C. jejuni killing, which correlated with enhanced LC3II conversion. These findings suggest that C. jejuni induces mTOR signaling as a way to avoid host-mediated killing, possibly by preventing adequate autophagy response.

Interestingly, S. typhimurium induced colitis was also inhibited by rapamycin exposure, suggesting that mTOR can be the target of other enteropathogenic microorganisms. mTOR has also been recently identified as a negative regulator of LPS-induced NF-κB signaling in monocytes, macrophage, and primary DC (Weichhart et al., 2008). Therefore, blocking mTOR signaling could enhance NF-κB signaling and production of inflammatory mediators involved in bacterial killing. However, the data disclosed herein do not support this hypothesis as EGFP expression (NF-κB activity) and some NF-κB-dependent inflammatory mediators are reduced in rapamycin-treated mice. While decreased activation of NF-κB (EGFP expression) and inflammatory gene expression in rapamycin-treated mice was observed herein, these observations are likely a secondary effect of the drug since direct NF-κB inhibitors failed to prevent campylobacteriosis (Lippert et al., 2009).

Although the majority of C. jejuni infected patients recover within 2 to 10 days without any specific treatment, a significant proportion develop severe clinical symptoms (fever, bloody diarrhea and abdominal cramps) which requires antibiotic treatment to shorten symptom durance (Allos et al., 2001). Recently, there are increasing concerns over C. jejuni resistance to antibiotics (Moore et al., 2006) and post-infectious sequelae such as Guillain-Barre Syndrome (GBS), inflammatory bowel disease (IBD) and irritable bowel syndrome (IBS). (Nachamkin, 2002; Gradel et al., 2009; Qin et al., 2010). Immune based prevention or intervention has attracted much attention as an alternative to antibiotic treatment. (Kirkpatrick and Tribble, 2010). Based on the novel findings presented herein, specific mTOR inhibitors (such as RAPAMUNE®, TORISEL®, AFINITOR® or ZORTRESS®) already approved for human use could represent a potential alternative to antibiotic-based therapy to treat campylobacteriosis.

In summary, this disclosure defines the role of mTOR in mediating the pro-inflammatory effect of C. jejuni infection. Neutrophil infiltration plays an active role in pathogenesis of C. jejuni infection and modulation of the cellular/molecular events leading to this process represent a new therapeutic arsenal to control campylobacteriosis.

### Materials and Methods for Examples 5-9

Mice and Tissue Processing

All animal protocols were approved by the Institutional Animal Care and Use Committee of the University of North Carolina at Chapel Hill (Chapel Hill, N.C., United States of America). Germ-free 8-12 week-old II10^{-/-}; NF-κBEGFP (129/SvEv; C57BL/6 mixed background), II10^{-/-} and II10^{-/-}; Rag2^{-/-} mice (129/SvEv) were transferred from germ-free isolators and immediately gavaged with a single dose of 10⁹ C. jejuni cfu/mouse (strain 81-176 (Korlath et al., 1985)) and sacrificed after up to 14 days as described previously (Sun et al., 2012). Specific pathogen free (SPF) housed Wt, II10^{-/-} and Pi3kγ^{-/-} (Sasaki et al., 2000) mice all on a 129/SvEv background were gavaged 10⁹ C. jejuni cfu/mouse one day after 7 day treatment with antibiotics cocktail (streptomycin 2 g/L, gentamycin 0.5 g/L, bacteriocin 1 g/L and ciprofloxacin 0.125 g/L) (Sun et al., 2012). To inhibit PI3K and PI3Kγ signaling, mice were i.p. injected daily with wortmannin (1.4 mg/kg; Fisher Scientific, Hampton, N.H., United States of America) and AS252424 (10 mg/kg; Cayman Chemical, Ann Arbor, Mich., United States of America), respectively. Tissue samples from colon, spleen, and mesenteric lymph nodes (MLN) were collected for protein, RNA, histology and C. jejuni culture assays as described previously (Sun et al., 2012). Histological images were acquired using a DP71 camera and DP Controller 3.1.1.276 (Olympus, Center Valley, Pa., United States of America), and intestinal inflammation was scored on a scale of 0 to 4 as described before (Lippert et al., 2009; Sun et al., 2012). Neutrophils in colonic tissues were identified based on morphological features using H&E stained sections and counted in 5 fields of view/mouse using a microscope. Data were expressed as average counts/mouse.

Neutrophil Depletion and IL-10 Receptor Blockade

II10^{-/-}; NF-κBEGFP mice were infected with C. jejuni and injected with anti-Gr1 antibody (i.p. 0.5 mg/mouse, at D0 and D3; clone: RB6-8C5; BioXcell, West Lebanon, N.H., United States of America) for 6 days to deplete neutrophil as described in a previous reports (Ribechini et al., 2009). A 6 day experimental time was selected instead of the typical 12 days because neutrophil depletion is less effective after 6 days. To block IL-10 signaling, antibiotic treated and C. jejuni infected Wt and Pi3kγ^{-/-} mice were injected with anti-IL-10R antibody (i.p. 0.5 mg/mouse, every 3 days; Clone: 1B1.3A; BioXcell, West Lebanon, N.H., United States of America) for 14 days as described (Bai et al., 2009). At the end of the experiment, mice were euthanized using CO₂ intoxication and death was ensured by performing cervical dislocation. Colons were resected and processed for H&E staining and colitis evaluation.

Western Blotting

A segment of the distal colon was lysed in 300 μl Laemmli buffer, homogenized and heated at 95° C. for 5 min. 20 μg of total protein were separated by SDS-PAGE and transferred to nitrocellulose membranes. Targeted protein was detected using enhanced chemiluminescence reaction (ECL) as described previously (Muhlbauer et al., 2008). Primary antibodies used were phosphor-AKT (S473), phosphor-p70S6K (T389), total AKT (all from Cell Signaling, Danvers, Mass., United States of America) and EGFP (Sigma, St. Louis, Mo., United States of America). The density of Western blot bands was quantified using ImageJ and data were normalized to total AKT control.

Neutrophil Isolation and Migration Assay

Neutrophils from the peripheral blood were isolated as described (Boxio et al., 2004). Briefly, blood (˜1 ml/mouse, 8 mice/group) from Wt and Pi3k^{-/-} mice was collected in 5 mM EDTA by cardiac puncture. The blood was diluted with 0.15M NaCl, loaded on a single layer of 69.2% Percoll and centrifuged at 1500×g for 20 min at room temperature. Neutrophils were recovered at the bottom layer of the gradients, and contaminating erythrocytes were lysed by hypotonic shock. Neutrophil purity was assessed using Wright-Giemsa staining and was found to be >98%. Cell migration assay was performed immediately after purification. A total of 5×10⁵ neutrophils were added in the top chamber of a Transwells (12 wells with 3 μm pore; Corning Inc., Corning, N.Y., United States of America) and CXCL-2 (250 ng/mL; R&D Systems, Minneapolis, Minn., United States of America) was added to the bottom wells. RMPI 1640 medium without CXCL-2 was used as a negative control. Transwells were then incubated for 2 h in humidified air and 5% CO₂. Neutrophils migrated into the bottom wells were imaged using a DP71 camera and DP Controller 3.1.1.276 (Olympus, Center Valley, Pa., United States of America) and enumerated using a hemocytometer (Sigma-Aldrich, St. Louis, Mo., United States of America). Cell viability was more than 95% as judged by trypan blue exclusion.

Enhanced GFP (EGFP) Macro-Imaging

Following infection and various treatment, II10^{-/-}; NF-κBEGFP mice were sacrificed, and the colon and cecum were removed and immediately imaged using a charge-coupled device camera in a light-tight imaging box with a dual-filtered light source and emission filters specific for EGFP (LT-99D2 Illumatools; Lightools Research, Encinitas, Calif., United States of America).

Fluorescence In Situ Hybridization (FISH)

Cy3-tagged 5′AGCTAACCACACCTTATACCG3′ (SEQ ID NO: 1) was used to probe the presence of C. jejuni in the intestinal tissue sections as previously described (Sun et al., 2012). Briefly, tissues were deparaffinized, hybridized with the probe, washed, mounted in DAPI medium and imaged using a Zeiss (Carl Zeiss SMT, Thornwood, N.Y., United States of America) LSM710 Spectral Confocal Laser Scanning Microscope system with ZEN 2008 software. Acquired images were analyzed using BioimageXD (Kankaanpaa et al., 2006).

Immunohistochemistry (IHC)

Neutrophils in intestinal tissue were detected using anti-myeloperoxidase (MPO) IHC as described previously (Sun et al., 2012). Briefly, intestinal tissue sections were deparaffinized, blocked and incubated with an anti-MPO antibody (1:400; Thermo Scientific, Hampton, N.H., United States of America) overnight. After incubation with anti-rabbit biotinylated antibody, ABC (Vectastain ABC Elite Kit, Vector Laboratories), DAB (Dako, Inc., Carpinteria, Calif., United States of America) and hematoxylin (Fisher Scientific, Hampton, N.H., United States of America), the sections were imaged on an OLYMPUS® microscope using DP 71 camera and DP Controller 3.1.1.276 (Olympus, Center Valley, Pa., United States of America).

C. Jejuni Quantification in Tissues

MLN and spleen were aseptically resected. Colon tissue was opened, resected and washed three times in sterile PBS. The tissues were weighed, homogenized in PBS, serially diluted and plated on Campylobacter selective blood plates (Remel, Thermo Scientific, Hampton, N.H., United States of America) for 48 h at 37° C. using the GasPak system (Becton Dickinson, Franklin Lakes, N.J., United States of America). C. jejuni colonies were counted and data presented as colony forming unit (cfu)/g tissue.

Real Time RT-PCR

Total RNA from intestinal tissues was extracted using TRIzol® kit (Invitrogen, Carlsbad, Calif., United States of America) following the manufacture's guide. cDNA was reverse-transcribed using M-MLV (Invitrogen). Proinflammatory 111β, Cxcl2, II17a and Tnfα mRNA expression levels were measured using SYBR® Green PCR Master mix (Applied Biosystems, Carlsbad, Calif., United States of America) on an ABI 7900HT Fast Real-Time PCR System and normalized to Gapdh. The PCR primers used were reported previously. (Sun et al., 2012). The PCR reactions were performed for 40 cycles according to the manufacturer's recommendation, and RNA fold changes were calculated using the ΔΔct method.

Primary Splenocyte Isolation and C. Jejuni Infection

Splenocytes were isolated as described previously. 13 Wt and Pi3kγ^{-/-} mice (8 to 12 weeks old) were sacrificed, spleens were resected and homogenized in RPMI 1640 medium supplemented with 2% fetal bovine serum (FBS), 2 mM L-glutamine and 50 μM 2-mercaptoethanol. Red blood cells were lysed, and cells were filtered through a 70 μm strainer, centrifuged, resuspended in the 2% FBS RPMI 1640 medium and plated at 1×10⁶ cells/well in 6-well plates. Cells were infected with C. jejuni (MOI 50) for 4 h in triplicates. Cells were then collected by centrifugation and lysed in TRIzol® (Invitrogen, Carlsbad, Calif., United States of America) for RNA extraction.

C. Jejuni Epithelial Cell Translocation Assay

Murine rectal carcinoma epithelial CMT-93 cells (1×10⁶) were plated onto 12-well Transwells (Corning Inc., Corning, N.Y., United States of America) in DMEM media containing 10% FBS and 2 mM L-glutamine. Upon reached confluency (monolayer), the medium was changed to 1% FBS medium and 10⁸ C. jejuni was added to the upper inserts in presence or absence of 10 μM AS252424. Aliquot of medium from bottom wells was collected every hr for 5 hrs, serially diluted and cultured on Remel (Remel, Thermo Scientific, Hampton, N.H., United States of America) plates as described before (Sun et al., 2012). Translocated C. jejuni was reported as CFU/ml at each time point.

Statistical Analysis

Values are shown as mean±SEM as indicated. Differences between groups were analyzed using the nonparametric Mann-Whitney U test. Experiments were considered statistically significant if P values <0.05. All calculations were performed using Prism 5.0 software.

### Example 5

### Innate Immune Cells Mediate C. Jejuni-Induced Colitis

Applicants have shown, using an antibody depletion approach, that C. jejuni 217 induced colitis in II10^{-/-} mice are CD4 independent (Sun et al., 2012). To further establish the role of innate and adaptive immunity in campylobacteriosis, germ-free II10^{-/-}; Rag2^{-/-} mice were utilized. Germ-free II10^{-/-} and II10^{-/-}; Rag2^{-/-} mice were transferred to specific pathogen free (SPF) housing and immediately gavaged with a single dose of C. jejuni (10⁹ CFU/mouse). After 12 days, as previously reported, C. jejuni induced severe intestinal inflammation in II10^{-/-} mice as showed by extensive immune cell infiltration, epithelial damage, goblet cell depletion and crypt hyperplasia and abscesses compared to uninfected mice (FIG. 12A). Interestingly, the absence of T and B cells did not impact the severity of colitis, as C. jejuni-infected II10^{-/-}; Rag2^{-/-} and II10^{-/-} mice developed comparable levels of intestinal inflammation. Similarly, C. jejuni-induced I11β, Cxcl2 and II17a mRNA accumulation was not significantly different between II10^{-/-}; Rag2^{-/-} and II10^{-/-} mice (FIG. 12B). Altogether, these observations indicate that C. jejuni-induced intestinal inflammation is predominantly mediated by innate immune cells during the early onset of campylobacteriosis (12 days).

### Example 6

### PI3K Signaling Mediates C. jejuni-Induced Colitis

As disclosed hereinabove, mTOR mediates C. jejuni-induced colitis. Since PI3K signaling is a potential upstream regulator of mTOR, experiments were designed and conducted to assess the role of this pathway in campylobacteriosis. C. jejuni-infected germ-free II10^{-/-}; NF-κBEGFP mice were i.p. injected daily with either vehicle (5% DMSO PBS) or with the pharmacological PI3K pan-inhibitor wortmannin (1.4 mg/kg body weight) for 12 days. As seen in FIG. 13A, C. jejuni-induced colitis and crypt abscesses were reduced in wortmannin-treated mice compared to vehicle-treated mice. Western blot analysis demonstrated reduction of C. jejuni-induced Akt phosphorylation (S473) in colonic lysates from wortmannin-treated, C. jejuni-infected mice (FIG. 13B). Moreover, C. jejuni-induced AKT phosphorylation (S473) in colonic lysates was not impaired in II10^{-/-}; Rag2^{-/-} mice, suggesting that lymphocytes are not the main contributor of Akt phosphorylation. To evaluate whole body transcriptional response, II10^{-/-}; NF-κBEGFP mice were infected with C. jejuni and the level of NF-κB-driven EGFP expression was determined. C. jejuni induced colonic EGFP expression in II10^{-/-}; NF-κBEGFP mice was attenuated in wortmannin-treated mice compared to vehicle-treated mice (FIG. 13B). In addition, wortmannin blocked C. jejuni-induced NF-κB dependent I11β, Cxcl2 and II17a mRNA accumulation by 50%, 77% and 78% respectively in II10^{-/-}; NF-κBEGFP mice compared to vehicle-treated, infected mice (FIG. 13C). These findings indicate that PI3K signaling is involved in C. jejuni-mediated intestinal inflammation.

### Example 7

### PI3Kγ Mediates C. jejuni-Induced Colitis

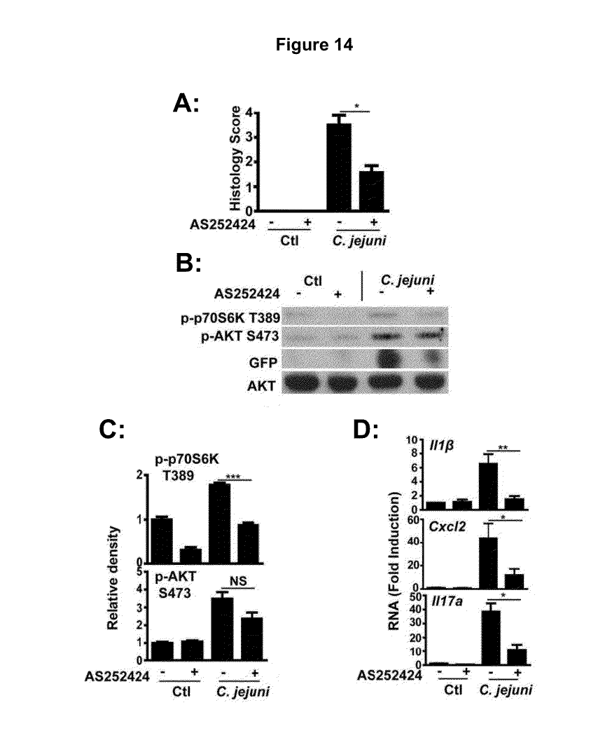

Since neutrophil infiltration and crypt abscesses are hallmarks of campylobacteriosis in both human and in the II10^{-/-} murine model (Sun et al., 2012; Blaser et al., 1980), the role of signal-induced neutrophil recruitment/activation in host pathogenesis was tested. Among PI3K family members, the class I B PI3Kγ has been implicated in leukocyte migration and activation. To establish the role of PI3Kγ in campylobacteriosis, germ-free II10^{-/-}; NFκBEGFP mice were gavaged with a single dose of C. jejuni (10⁹ CFU/mouse) and i.p. injected daily with PI3Kγ specific inhibitor AS252424 (10 mg/kg body weight) or vehicle control (5% DMSO PBS) for 6 days. Interestingly, C. jejuni-induced colitis was reduced in AS252424-treated II10^{-/-}; NF-κBEGFP mice compared to vehicle-treated mice (FIG. 14A). Western blot analysis (FIG. 14B) demonstrated a modest reduction (FIG. 14C) of AKT phosphorylation (S473) but an evident attenuation (34%) of p70S6K phosphorylation (T389) in colonic extracts from AS252424-treated, C. jejuni-infected II10^{-/-}; NF-κBEGFP mice. In addition, induction of EGFP expression (NF-κB activity) in the colon of C. jejuni infected II10^{-/-}; NF-κBEGFP mice was reduced in AS252424-treated mice compared to control vehicle-treated mice (FIGS. 14B-14C). Next, the impact of PI3Kγ on expression of NF-κB-dependent proinflammatory mediators involved in bacterial host responses was examined. AS252424 treatment blocked C. jejuni-induced I11β, Cxcl2 and II17a mRNA accumulation by 77%, 73% and 72%, respectively, compared to vehicle treated, infected II10^{-/-}; NF-κBEGFP mice (FIG. 14D).

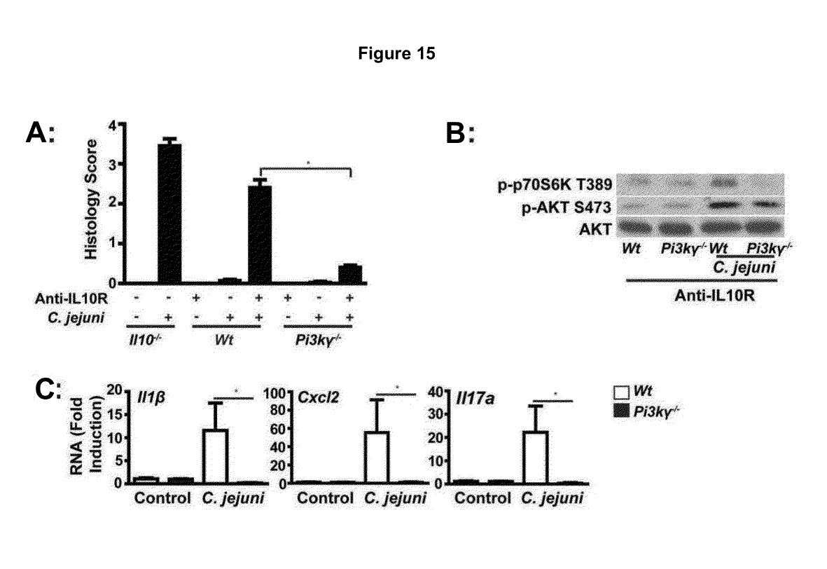

Next, to gain specificity over the pharmacological targeting approach, Pi3kγ^{-/-} mice were utilized. Antibiotic treatment has been shown to enhance C. jejuni colonization in Wt mice (Sun et al., 2012). Interestingly, antibiotic-treated II10^{-/-} mice displayed severe colitis at 2 weeks, but antibiotic-treated SPF Wt and Pi3kγ^{-/-} mice were resistant to C. jejuni induced colitis (FIG. 15A). To enhance susceptibility of Wt and Pi3kγ^{-/-} mice to C. jejuni induced colitis, the II10 knockout phenotype was emulated by using an antibody blocking the IL-10 receptor (IL-10R). Antibiotic-treated Wt and Pi3kγ^{-/-} mice were gavaged with C. jejuni and then i.p. injected with anti-IL-10R antibody (500 μg/mouse) every 3 days for 2 weeks. As predicted, anti-IL-10R-treated Wt mice developed colitis following C. jejuni infection, albeit to a slightly lower extent than II10^{-/-} mice (FIG. 15A). In agreement with the pharmacologic studies, C. jejuni-induced intestinal inflammation was strongly attenuated in anti-IL-10R-treated Pi3kγ^{-/-} mice compared to anti-IL-10R-treated Wt mice.

No evidence of intestinal inflammation was observed in uninfected Wt mice treated with anti-IL-10R antibody alone. Western blot analysis demonstrated a slight reduction of AKT phosphorylation (S473) but a strong blockade (68%) of p70S6K phosphorylation (T389) in intestinal lysates from IL-10R-blocked, C. jejuni-infected Pi3kγ^{-/-} mice compared to Wt mice (FIG. 15B). In accordance with the histological score, the absence of PI3Kγ strongly reduced C. jejuni-induced II10, Cxcl2 and II17a mRNA accumulation (98.3%, 98.2% and 98.4%, respectively) in IL-10R-blocked Pi3kγ^{-/-} mice, compared to treated Wt mice (FIG. 15C). To determine whether absence of PI3Kγ signaling is directly responsible for decreased C. jejuni-induced inflammatory gene expression, splenocytes from Wt and Pi3kγ^{-/-} mice were isolated. Interestingly, C. jejuni-induced II10, Cxcl2, 1117a and Tnfa mRNA expression was comparable between splenocytes obtained from Pi3kγ^{-/-} and Wt mice. These results suggest that PI3Kγ signaling does not directly regulate C. jejuni induced proinflammatory gene expression.

### Example 8

### PI3Kγ Mediates C. jejuni Invasion

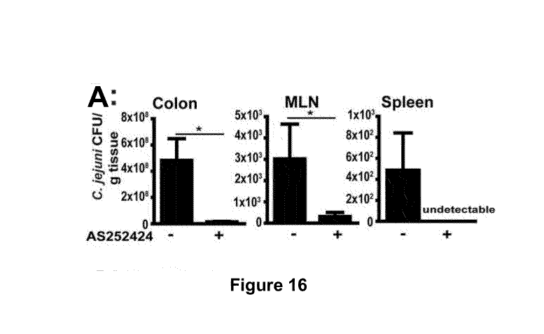

Since C. jejuni is an invasive intestinal pathogenic bacterium, the impact of PI3Kγ signaling on C. jejuni invasion into intra- and extra-intestinal tissues was investigated next. Following infection and treatment with the PI3Kγ inhibitor AS252424, C. jejuni DNA was visualized in the colon of II10^{-/-}; NF-κBEGFP mice using fluorescence in situ hybridization (FISH) and confocal microscopy imaging. Remarkably, while C. jejuni was abundant in inflamed crypts and lamina propria of vehicle-treated mice, the bacterium was barely detectable in AS252424-treated mice. To assess the amount of viable C. jejuni in intestinal and extra-intestinal tissues, samples from the colon, spleen and MLN were aseptically collected, and C. jejuni were enumerated on Remel Campylobacter selective plates. Consistent with FISH results, AS252424 treatment decreased the amount of viable C. jejuni in colon and MLN by 97% and 90%, respectively, compared to C. jejuni-infected, vehicle-treated mice (FIG. 16A). Moreover, AS252424 treatment strongly reduced the levels of viable C. jejuni in the spleen, compared to vehicle-treated, infected mice.

Again, to confirm these findings from pharmacologic studies, Pi3kγ^{-/-} mice treated with IL-10R blocking antibody were infected and C. jejuni invasion was assessed using FISH. C. jejuni was detected deeply inside the intestinal section of anti-IL-10R-treated Wt mice, whereas anti-IL-10R-treated Pi3kγ^{-/-} mice exhibited a strong reduction in bacterial invasion into colonic tissues. To determine whether PI3Kγ signaling derived from epithelial cells directly affects C. jejuni invasion, a monolayer of murine colonic CMT-93 cells was infected with C. jejuni in the presence or absence of AS252424 and bacterial translocation was measured using a transwell culture system. PI3Kγ inhibition did not prevent C. jejuni translocation. Taken together, these results demonstrate that C. jejuni invasion into intestinal and extra intestinal tissue is dependent upon functional PI3Kγ signaling, likely originating from immune cells.

### Example 9

### Neutrophil Infiltration Promotes C. jejuni-Induced Colitis

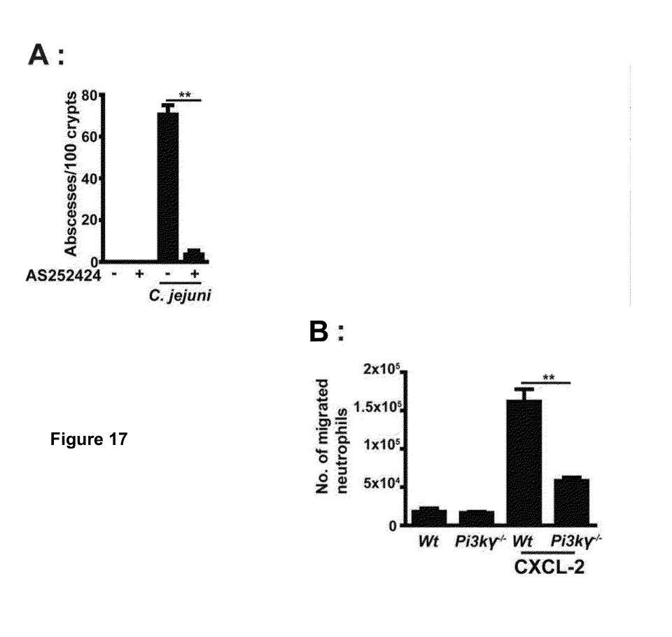

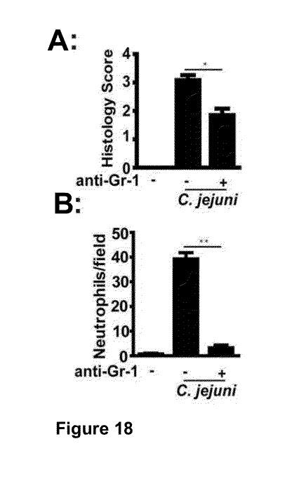

Crypt abscesses and neutrophil infiltration is predominant in C. jejuni-infected II10^{-/-} mice (Sun et al., 2012). As seen in FIG. 17A, C. jejuni infection of II10^{-/-} mice induced an average of greater than 71 colonic crypt abscesses per 100 crypts. Remarkably, C. jejuni-induced crypt abscesses were reduced by 95% in AS252424-treated II10^{-/-} mice compared to vehicle-treated mice. In accordance with this finding, MPO staining revealed that C. jejuni-induced neutrophil infiltration into colonic tissues was strongly reduced in the presence of AS252424. Since PI3Kγ is implicated in neutrophil migration (Sasaki et al., 2000), peripheral blood neutrophil motility was evaluated in response to the chemokine CXCL-2 using a transwell migration assay. Migration was reduced by 64% in neutrophils isolated from Pi3kγ^{-/-} mice compared to Wt cells (FIG. 17B). Together these observations demonstrate that suppression of C. jejuni-induced colitis by pharmacologic inhibition of PI3Kγ is associated with an impaired neutrophil migration/infiltration and subsequent crypt abscess formation. To directly assess the role of neutrophils in C. jejuni-induced colitis, these cells were depleted using an anti-Gr-1 antibody (i.p. every 3 days for 6 days). Depletion of neutrophils attenuated C. jejuni-induced colitis in II10^{-/-} mice, as demonstrated by the significant reduction of histological scores (FIG. 18A), which correlated with reduced MPO staining. Numbers of colonic neutrophils were reduced by more than 92% in anti-Gr-1 antibody-treated, C. jejuni infected mice compared to untreated mice (FIG. 18B). To determine the effect of neutrophil depletion on C. jejuni invasion, C. jejuni presence in colon tissues was visualized using FISH assay. Interestingly, although neutrophils were depleted, C. jejuni invasion into the colon was strongly attenuated, suggesting that other immune cells such as macrophages and dendritic cells are important in eliminating invading C. jejuni. Collectively, these results demonstrate that PI3Kγ signaling is essential for C. jejuni induced intestinal inflammation, by modulating neutrophil infiltration and migration into the intestinal tissues.

### Discussion of Examples 5-9

Examples 1-4 show that C. jejuni induces intestinal inflammation through mTOR signaling, a downstream target of the PI3K pathway, which is associated with neutrophil infiltration and tissue damage (Sun et al., 2012). In addition, because PI3Ks form a large family of kinases, the subunit responsible for neutrophil activation and migration following C. jejuni infection is unclear. Thus, Examples 5-8 uncover the role of innate immune cells, especially neutrophils in C. jejuni-induced intestinal inflammation. In addition, the instant disclosure identifies PI3Kγ signaling as playing a role in campylobacteriosis.

Among the large family of PI3Ks, PI3Kγ is predominantly expressed in immune cells (Li et al., 2000; Sasaki et al., 2000). Disruption of PI3Kγ attenuates E. coli-induced lung injury resulting from neutrophil infiltration (Ong et al., 2005). Similarly, a reduction of neutrophil-mediated rheumatoid arthritis is observed in Pi3kγ^{-/-} mice (Camps et al., 2005). The above disclosure demonstrates that C. jejuni-induced colitis can be alleviated by inactivation of PI3Kγ signaling using either pharmacological or genetic manipulation.

Using FISH and culture assays, it was observed that PI3Kγ signaling promotes C. jejuni invasion into colon, MLN and spleen of II10^{-/-} mice. Immunohistochemistry assays revealed massive infiltration of neutrophils into the colon following C. jejuni infection, an effect attenuated by inactivation of PI3Kγ. Moreover, depletion of neutrophils using anti-Gr1 antibody reduced C. jejuni induced intestinal inflammation by ˜40%, an effect comparable to inactivation of PI3Kγ. Taken together, these findings highlight the role of PI3Kγ signaling and neutrophils in C. jejuni pathogenesis.

The contribution of innate and adaptive immune cells in host response to C. jejuni infection is not well understood. Adaptive immunity has been documented to protect the host against C. jejuni-induced diarrhea and intestinal inflammation in CD4 deficient HIV patients (Snijders et al., 1997). On the other hand, the plasma of C. jejuni infected patients have been shown to contain anti-ganglioside 1 IgG (Oomes et al., 1995) mimicry between the core lipooligosaccharides of C. jejuni and human gangliosides which can be associated with the development of Guillain-Barre Syndrome (Nachamkin, 2002). Interestingly, adaptive immunity might not been essential for early intestinal inflammation as C. jejuni induced colitis is similar between II10^{-/-}; Rag2^{-/-} mice and II10^{-/-} mice. This finding suggests that innate immune cells are at least one of the cellular components responsible for the acute state (about 12 days) of campylobacteriosis. In addition, IHC analysis in conjunction with cell migration and depletion studies strongly point neutrophils playing a role in campylobacteriosis. Therefore, although persistent C. jejuni infection triggers an adaptive immune response, the initial responses and associated tissue damage is mediated by neutrophils.

Following enteric bacterial infection, neutrophils are rapidly recruited into intestinal tissues where they eliminate microorganisms through phagocytosis and degranulation mediated bacterial killing (Brinkmann et al., 2004). However, overzealous neutrophil recruitment into a defined location like intestinal crypts often leads to significant host tissue damage. Neutrophil-induced tissue damage has been reported in non-infectious diseases such as IBD (Chin et al., 2006), lung injury (Ong et al., 2005) and arthritis (Camps et al., 2005). In C. jejuni-infected patients, histological assessment of intestinal tissues has revealed neutrophil infiltration and crypt abscesses (van Spreeuwel et al., 1985). Using transmission electron microscopy analysis, it has been shown that crypt microvilli are virtually destroyed by accumulated neutrophils (Sun et al., 2012). The presently disclosed subject matter demonstrates that antibody-mediated depletion of neutrophils diminishes intestinal inflammation and strongly decreases crypt abscess formation.

Data from the presently disclosed subject matter indicates that neutrophils are involved in C. jejuni mediated pathogenesis. As for the molecular events leading to their recruitment into intestinal crypts, the presently disclosed subject matter shows a strong induction of the chemokine Cxcl2 in colonic lysates of C. jejuni infected II10^{-/-} mice. In vitro experiments indicate that intestinal epithelial cells (Lippert et al., 2009) and splenocytes up-regulate Cxcl2 gene expression following C. jejuni infection. As such, without being bound by any one particular theory, it is believed that C. jejuni invasion leads to the secretion of various chemo-attractants including CXCL-2, from immune and non immune cells, which then promote recruitment of neutrophils. Interestingly, C. jejuni induced proinflammatory gene expression including Cxcl2 is comparable in splenocytes isolated from Pi3kγ^{-/-} and Wt mice and blocking PI3Kγ does not attenuate C. jejuni invasion through CMT-93 epithelial monolayer. Thus, it is concluded that PI3Kγ signaling predominantly mediates its inhibitory effect through regulation of neutrophil migration.

Interestingly, although neutrophils most likely participate in the removal of invading C. jejuni, FISH assay showed a strong decrease of the bacterium in colonic tissues of GR-1-treated mice. This finding suggests that other innate cells such as macrophages and dendritic cells are important for C. jejuni eradication in the colon. In this scenario, the beneficial impact of neutrophils in C. jejuni elimination is outweighed by the tissue destructive capacity of these innate cells and associated damage to the epithelial barrier. It is likely that C. jejuni located in the luminal compartment profits from this impaired barrier function to further invade the colonic tissues.

Presently, the primary treatment for campylobacteriosis resorts to antibiotics. However, antibiotic treatment is constrained by multiple factors, including minimal effectiveness in the late course of disease, a negligible reduction in disease duration (1.5 days), increased antibiotic resistance, and the risk of harmful eradication of normal flora (Ternhag et al., 2007). Thus alternatives to antibiotics are imperative for treating infectious enteric pathogens, and immunotherapy targeting specific signaling pathways such as PI3Kγ may provide such an alternative.

In summary, these Examples define for the first time the role of PI3Kγ in mediating the pro inflammatory effects of C. jejuni infection. PI3Kγ mediated neutrophil infiltration plays an active role in the pathogenesis of C. jejuni infection. Accordingly, modulation of the cellular/molecular events leading to this process represents a new therapeutic approach to control campylobacteriosis.

### REFERENCES

1. Allos B M. Campylobacter jejuni Infections: update on emerging issues and trends. Clin Infect Dis 2001; 32:1201-6.
2. Bai, F., T. Town, F. Qian, P. Wang, M. Kamanaka, T. M. Connolly, D. Gate, R. R. Montgomery, R. A. Flavell, and E. Fikrig. 2009. IL-10 signaling blockade controls murine West Nile virus infection. PLoS Pathog 5:e1000610.
3. Blaser M J. Epidemiologic and clinical features of Campylobacter jejuni infections. J Infect Dis 1997; 176 Suppl 2:S103-5.
4. Blaser, M. J., R. B. Parsons, and W. L. Wang. 1980. Acute colitis caused by Campylobacter fetus ss. jejuni. Gastroenterology 78:448-453.
5. Boxio, R., C. Bossenmeyer-Pourie, N. Steinckwich, C. Dournon, and O. Nusse. 2004. Mouse bone marrow contains large numbers of functionally competent neutrophils. J Leukoc Biol 75:604-611.
6. Brinkmann, V., U. Reichard, C. Goosmann, B. Fauler, Y. Uhlemann, D. S. Weiss, 635 Y. Weinrauch, and A. Zychlinsky. 2004. Neutrophil extracellular traps kill bacteria. Science 303:1532-1535.
7. Camps, M., T. Ruckle, H. Ji, V. Ardissone, F. Rintelen, J. Shaw, C. Ferrandi, C. Chabert, C. Gillieron, B. Francon, T. Martin, D. Gretener, D. Perrin, D. Leroy, P. A. Vitte, E. Hirsch, M. P. Wymann, R. Cirillo, M. K. Schwarz, and C. Rommel. 2005. Blockade of PI3Kgamma suppresses joint inflammation and damage in mouse models of rheumatoid arthritis. Nat Med 11:936-943.
8. CDC. 2012. Incidence of laboratory-confirmed bacterial and parasitic infections, and postdiarrheal hemolytic uremic syndrome (HUS), by year and pathogen, Foodborne Diseases Active Surveillance Network (FoodNet), United States, 1996-2011. Centers for Disease Control and Prevention. http://www.cdc.gov/foodnet/data/trends/tables/table2a-b.html#table-2b.
9. CDC. Campylobacter: General Information. Available at http://www.cdc.gov/nczved/divisions/dfbmd/diseases/campvlobacter/. Accessed Oct. 19, 2010, 2010.
10. Chin, A. C., and C. A. Parkos. 2006. Neutrophil transepithelial migration and epithelial barrier function in IBD: potential targets for inhibiting neutrophil trafficking. Ann NY Acad Sci 1072:276-287.
11. Chou R C, Kim N D, Sadik C D, Seung E, Lan Y, Byrne M H, Haribabu B, Iwakura Y, Luster A D. Lipid-cytokine-chemokine cascade drives neutrophil recruitment in a murine model of inflammatory arthritis. Immunity 2010; 33:266-78.
12. Ferguson, G. J., L. Milne, S. Kulkarni, T. Sasaki, S. Walker, S. Andrews, T. Crabbe, P. Finan, G. Jones, S. Jackson, M. Camps, C. Rommel, M. Wymann, E. Hirsch, P. Hawkins, and L. Stephens. 2007. PI(3)Kgamma has an important context-dependent role in neutrophil chemokinesis. Nat Cell Biol 9:86-91.
13. Fire et al., Nature 391:806-811, 1998
14. Fire, Trends Genet 15:358-363, 1999
15. Fouts, D. E., E. F. Mongodin, R. E. Mandrell, W. G. Miller, D. A. Rasko, J. Ravel, L. M. Brinkac, R. T. DeBoy, C. T. Parker, S. C. Daugherty, R. J. Dodson, A. S. Durkin, R. Madupu, S. A. Sullivan, J. U. Shetty, M. A. Ayodeji, A. Shvartsbeyn, M. C. Schatz, J. H. Badger, C. M. Fraser, and K. E. Nelson. 2005. Major structural differences and novel potential virulence mechanisms from the genomes of multiple campylobacter species. PLoS Biol 3:e15.
16. Gomez-Cambronero J, Horn J, Paul C C, Baumann M A. Granulocyte-macrophage colony-stimulating factor is a chemoattractant cytokine for human neutrophils: involvement of the ribosomal p70 S6 kinase signaling pathway. J Immunol 2003; 171:6846-55.
17. Gradel K O, Nielsen H L, Schønheyder H C, Ejlertsen T, Kristensen B, Nielsen H. Increased Short- and Long-Term Risk of Inflammatory Bowel Disease After Salmonella or Campylobacter Gastroenteritis. Gastroenterology 2009; 137:495-501.
18. Gradel, K. O., H. L. Nielsen, H. C. Schønheyder, T. Ejlertsen, B. Kristensen, and H. Nielsen. 2009. Increased Short- and Long-Term Risk of Inflammatory Bowel Disease After Salmonella or Campylobacter Gastroenteritis. Gastroenterology 137:495-501.
19. Gunzl, P., and G. Schabbauer. 2008. Recent advances in the genetic analysis of PTEN and PI3K innate immune properties. Immunobiology 213:759-765.
20. Gutierrez M G, Master S S, Singh S B, Taylor G A, Colombo M I, Deretic V. Autophagy Is a Defense Mechanism Inhibiting BCG and Mycobacterium tuberculosis Survival in Infected Macrophages. Cell 2004; 119:753-766.
21. Hapfelmeier S, Stecher B, Barthel M, Kremer M, Muller A J, Heikenwalder M, Stallmach T, Hensel M, Pfeffer K, Akira S, Hardt W D. The Salmonella pathogenicity island (SPI)-2 and SPI-1 type III secretion systems allow Salmonella serovar typhimurium to trigger colitis via MyD88-dependent and MyD88-independent mechanisms. J Immunol 2005; 174:1675-85.
22. Hofreuter, D., J. Tsai, R. O. Watson, V. Novik, B. Altman, M. Benitez, C. Clark, C. Perbost, T. Jarvie, L. Du, and J. E. Galan. 2006. Unique features of a highly pathogenic Campylobacter jejuni strain. Infect Immun 74:4694-4707.
23. Inoki K, Li Y, Zhu T, Wu J, Guan K-L. TSC2 is phosphorylated and inhibited by Akt and suppresses mTOR signalling. Nature Cell Biology 2002; 4:648-657.
24. Janes M R, Fruman D A. Immune Regulation by Rapamycin: Moving Beyond T Cells. Science Signaling 2009; 2:pe25-pe25.
25. Kankaanpää P, Pahajoki K, Marjomäki V, Heino J, White D. BiolmageXD-New Open Source Free Software for the Processing, Analysis and Visualization of Multidimensional Microscopic Images. Microscopy Today 2006; 14:12-16.
26. Kim S C, Tonkonogy S L, Albright C A, Tsang J, Balish E J, Braun J, Huycke M M, Sartor R B. Variable phenotypes of enterocolitis in interleukin 10-deficient mice monoassociated with two different commensal bacteria. Gastroenterology 2005; 128:891-906.
27. Kim S R, Lee K S, Park S J, Min K H, Lee K Y, Choe Y H, Lee Y R, Kim J S, Hong S J, Lee Y C. PTEN down-regulates IL-17 expression in a murine model of toluene diisocyanate-induced airway disease. J Immunol 2007; 179:6820-9.
28. Kirkpatrick B D, Tribble D R. Update on human Campylobacter jejuni infections. Curr Opin Gastroenterol. 2010/12/03 ed, 2010.
29. Korlath J A, Osterholm M T, Judy L A, Forfang J C, Robinson R A. A point-source outbreak of campylobacteriosis associated with consumption of raw milk. J Infect Dis 1985; 152:592-6.
30. Koyasu, S. 2003. The role of PI3K in immune cells. Nat Immunol 4:313-319.
31. Law B F, Adriance S M, Joens L A. Comparison of in vitro virulence factors of Campylobacter jejuni to in vivo lesion production. Foodborne Pathog Dis 2009; 6:377-85.
32. Li, Z., H. Jiang, W. Xie, Z. Zhang, A. V. Smrcka, and D. Wu. 2000. Roles of 587 PLC-beta2 and -beta3 and PI3Kgamma in chemoattractant-mediated signal transduction. Science 287:1046-1049.
33. Lippert E, Karrasch T, Sun X, Allard B, Herfarth H H, Threadgill D, Jobin C. Gnotobiotic IL-10; NF-kappaB mice develop rapid and severe colitis following Campylobacter jejuni infection. PLoS One 2009; 4:e7413.
34. Mansfield, L., J. Patterson, B. Fierro, A. Murphy, V. Rathinam, J. Kopper, N. 575 Barbu, T. Onifade, and J. Bell. 2008. Genetic background of IL-10−/− mice alters host-pathogen interactions with Campylobacter jejuni and influences disease phenotype. Microbial Pathogenesis 45:241-257.
35. Moore J E, Barton M D, Blair I S, Corcoran D, Dooley J S, Fanning S, Kempf I, Lastovica A J, Lowery C J, Matsuda M, McDowell D A, McMahon A, Millar B C, Rao J R, Rooney P J, Seal B S, Snelling W J, Tolba O. The epidemiology of antibiotic resistance in Campylobacter. Microbes Infect 2006; 8:1955-66.
36. Mortensen, N. P., M. L. Kuijf, C. W. Ang, P. Schiellerup, K. A. Krogfelt, B. C. Jacobs, A. van Belkum, H. P. Endtz, and M. P. Bergman. 2009. Sialylation of Campylobacter jejuni lipo-oligosaccharides is associated with severe gastro541 enteritis and reactive arthritis. Microbes Infect 11:988-994.
37. Muhlbauer, M., P. M. Chilton, T. C. Mitchell, and C. Jobin. 2008. Impaired Bcl3 Up-regulation Leads to Enhanced Lipopolysaccharide-induced Interleukin (IL)-23P19 Gene Expression in IL-10−/− Mice. Journal of Biological Chemistry 611 283:14182-14189.
38. Nachamkin, I. 2002. Chronic effects of Campylobacter infection. Microbes Infect 4:399-403.
39. Nemelka K W, Brown A W, Wallace S M, Jones E, Asher L V, Pattarini D, Applebee L, Gilliland T C, Jr., Guerry P, Baqar S. Immune response to and histopathology of Campylobacter jejuni infection in ferrets (Mustela putorius furo). Comp Med 2009; 59:363-71.
40. Ong, E., X. P. Gao, D. Predescu, M. Broman, and A. B. Malik. 2005. Role of phosphatidylinositol 3-kinase-gamma in mediating lung neutrophil sequestration and vascular injury induced by E. coli sepsis. Am J Physiol Lung Cell Mol Physiol 289:L1094-1103.
41. Oomes, P. G., B. C. Jacobs, M. P. Hazenberg, J. R. Banffer, and F. G. van der Meche. 1995. Anti-GM1 IgG antibodies and Campylobacter bacteria in Guillain-Barre syndrome: evidence of molecular mimicry. Ann Neurol 38:170-175.
42. Poppert S, Haas M, Yildiz T, Alter T, Bartel E, Fricke U, Essig A. Identification of Thermotolerant Campylobacter Species by Fluorescence In Situ Hybridization. Journal of Clinical Microbiology 2008; 46:2133-2136.
43.
44. Qin, H. Y., J. C. Wu, X. D. Tong, J. J. Sung, H. X. Xu, and Z. X. Bian. 2010. 547 Systematic review of animal models of post-infectious/post-inflammatory irritable bowel syndrome. In J Gastroenterol, 2010/09/18 ed. DOI: 10.1007/s00535-00010-00321-00536.
45. Ribechini, E., P. J. M. Leenen, and M. B. Lutz. 2009. Gr-1 antibody induces STAT signaling, macrophage marker expression and abrogation of myeloid603 ved suppressor cell activity in BM cells. European Journal of Immunology 39:3538-3551.
46. Russell R G, Blaser M J, Sarmiento J I, Fox J. Experimental Campylobacter jejuni infection in Macaca nemestrina. Infect Immun 1989; 57:1438-44.
47. Rydstrom, A., and M. J. Wick. 2009. Monocyte and neutrophil recruitment during 581 oral Salmonella infection is driven by MyD88-derived chemokines. Eur J Immunol 39:3019-3030.
48. Sasaki, T., J. Irie-Sasaki, R. G. Jones, A. J. Oliveira-dos-Santos, W. L. Stanford, B. Bolon, A. Wakeham, A. Itie, D. Bouchard, I. Kozieradzki, N. Joza, T. W. Mak, P. S. Ohashi, A. Suzuki, and J. M. Penninger. 2000. Function of PI3Kgamma in thymocyte development, T cell activation, and neutrophil migration. Science 593 287:1040-1046.
49. Shibata K, Yamada H, Hara H, Kishihara K, Yoshikai Y. Resident Vdelta1+gammadelta T cells control early infiltration of neutrophils after Escherichia coli infection via IL-17 production. J Immunol 2007; 178:4466-72.
50. Sieve A N, Meeks K D, Bodhankar S, Lee S, Kolls J K, Simecka J W, Berg R E. A novel IL-17-dependent mechanism of cross protection: Respiratory infection with mycoplasma protects against a secondary listeria infection. European Journal of Immunology 2009; 39:426-438.
51. Snijders, F., E. J. Kuijper, B. de Wever, L. van der Hoek, S. A. Danner, and J. 629 Dankert. 1997. Prevalence of Campylobacter-associated diarrhea among patients infected with human immunodeficiency virus. Clin Infect Dis 24:1107-1113.
52. Sun, X., D. Threadgill, and C. Jobin. 2012. Campylobacter jejuni Induces Colitis Through Activation of Mammalian Target of Rapamycin Signaling. Gastroenterology 142:86-95 e85.
53. Ternhag, A., T. Asikainen, J. Giesecke, and K. Ekdahl. 2007. A meta-analysis on the effects of antibiotic treatment on duration of symptoms caused by infection Campylobacter species. Clin Infect Dis 44:696-700.
54. U.S. Pat. No. 5,580,722
55. U.S. Pat. No. 5,846,720
56. U.S. Pat. No. 6,506,559
57. Valenza, G., M. Frosch, and M. Abele-Horn. 2010. Antimicrobial susceptibility of clinical Campylobacter isolates collected at a German university hospital during the period 2006-2008. Scand J Infect Dis 42:57-60.
58. van den Bruele, T., P. E. Mourad-Baars, E. C. Claas, R. N. van der Plas, E. J. Kuijper, and R. G. Bredius. 2010. Campylobacter jejuni bacteremia and Helicobacter pylori in a patient with X-linked agammaglobulinemia. Eur J Clin 553 Microbiol Infect Dis 29:1315-1319.
59. van Spreeuwel J P, Duursma G C, Meijer C J, Bax R, Rosekrans P C, Lindeman J. Campylobacter colitis: histological immunohistochemical and ultrastructural findings. Gut 1985; 26:945-951.
60. Venable, D et al. Phosphoinositide 3-Kinase Gamma (PI3Kγ) Inhibitors for the Treatment of Inflammation and Autoimmune Disease. Recent Patents on Inflammation and Allergy Drug Discovery 2010, 4:1-15.
61. Weichhart T, Saemann M. The PI3K/Akt/mTOR pathway in innate immune cells: emerging therapeutic applications. Ann Rheum Dis 2008:67(Suppl III):iii70-iii74.
62. Weichhart T, Costantino G, Poglitsch M, Rosner M, Zeyda M, Stuhlmeier K, Kolbe T, Stulnig T, Horl W, Hengstschlager M. The TSC-mTOR Signaling Pathway Regulates the Innate Inflammatory Response. Immunity 2008; 29:565-577.
63. Wullschleger S, Loewith R, Hall M N. TOR signaling in growth and metabolism. Cell 2006; 124:471-84.
64. Xu S, Cao X. Interleukin-17 and its expanding biological functions. Cellular and Molecular Immunology 2010; 7:164-174.
65. Ye P, Rodriguez F H, Kanaly S, Stocking K L, Schurr J, Schwarzenberger P, Oliver P, Huang W, Zhang P, Zhang J, Shellito J E, Bagby G J, Nelson S, Charrier K, Peschon J J, Kolls J K. Requirement of interleukin 17 receptor signaling for lung CXC chemokine and granulocyte colony-stimulating factor expression, neutrophil recruitment, and host defense. J Exp Med 2001; 194:519-27.
66. Young, K. T., L. M. Davis, and V. J. Dirita. 2007. Campylobacter jejuni: molecular biology and pathogenesis. Nat Rev Microbiol 5:665-679.
67. Yuki N, Susuki K, Koga M, Nishimoto Y, Odaka M, Hirata K, Taguchi K, Miyatake T, Furukawa K, Kobata T, Yamada M. Carbohydrate mimicry between human ganglioside GM1 and Campylobacter jejuni lipooligosaccharide causes Guillain-Barre syndrome. Proc Natl Acad Sci USA 2004; 101:11404-9.

The Sequence Listing is provided herewith as an ASCII .txt file entitled Sequence Listing, 421-297-2_{—}5T25, created Feb. 25, 2013, 2100 bytes (21 kilobytes), and is incorporated here by reference in its entirety.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating enteritis in a subject, the method comprising:
providing a subject suffering from enteritis; and
administering to the subject a composition comprising a compound capable of modulating a component of a PI3K pathway,
wherein the enteritis is treated.

2. The method of claim 1, wherein a causative agent of the enteritis is selected from the group consisting of Campylobacter jejuni, Salmonella typhimurium, Enteropathogenic Escherichia coli and Shigella.

3. The method of claim 2, wherein the subject is suffering from campylobacteriosis.

4. The method of claim 1, wherein the compound capable of modulating a component of the PI3K pathway comprises an inhibitor of mammalian target of rapamycin (mTOR).

5. The method of claim 4, wherein the inhibitor of mTOR is rapamycin, rapamycin derivatives or analogues.

6. The method of claim 4, wherein the inhibitor of mTOR is Rapamune, Torisel, Afinitor or Zortress.

7. The method of claim 1, wherein the compound capable of modulating a component of the PI3K pathway comprises an inhibitor of PI3K.

8. The method of claim 7, wherein the inhibitor of PI3K is wortmannin.

9. The method of claim 1, wherein the compound capable of modulating a component of the PI3K pathway comprises an inhibitor of PI3Kγ.

10. The method of claim 9, wherein the inhibitor of PI3Kγ is selected from the group consisting of AS252424, thiazolidinones, thiazolidinones, and 2-aminothiazoles.

11. The method of claim 1, wherein treating the enteritis comprises reduced intestinal inflammation or increased bacterial clearance.

12. The method of claim 1, wherein the subject is a human.

13. A method of identifying an agent to treat enteritis, the method comprising:
providing a test sample comprising a polypeptide of a PI3K pathway;
administering a test molecule to the test sample; and
determining the effect of the test molecule on the activity of the polypeptide of a PI3K pathway.

14. The method of claim 13, wherein the polypeptide of the PI3K pathway comprises mTOR complex 1 or mTOR complex 2.

15. The method of claim 13, wherein the polypeptide of the PI3K pathway comprises PI3Kγ.

16. The method of claim 13, wherein the effect of the test molecule on the activity of the polypeptide of the PI3K pathway is a modulatory effect.

17. The method of claim 16, wherein the modulatory effect on the polypeptide of the PI3K pathway is an inhibition of a signaling activity of the PI3K polypeptide.

18. A therapeutic composition to treat enteritis in a subject, the therapeutic composition comprising:
a compound capable of modulating a component of a PI3K pathway; and
a pharmaceutically acceptable carrier.

19. The therapeutic composition of claim 18, wherein the compound capable of modulating a component of the PI3K pathway comprises an inhibitor of mTOR.

20. The therapeutic composition of claim 19, wherein the inhibitor of mTOR is rapamycin, rapamycin derivatives or analogues.

21. The therapeutic composition of claim 18, wherein the compound capable of modulating a component of the PI3K pathway comprises an inhibitor of PI3K.

22. The therapeutic composition of claim 21, wherein the inhibitor of PI3K is wortmannin.

23. The therapeutic composition of claim 18, wherein the compound capable of modulating a component of the PI3K pathway comprises an inhibitor of PI3Kγ.

24. The therapeutic composition of claim 23, wherein the inhibitor of PI3Kγ is selected from the group consisting of AS252424, thiazolidinones, thiazolidinones, and 2-aminothiazoles.
